(19) United States
(12) Patent Application Publication
(10) Pub. No.: US 2025/0145679 A1
(43) Pub. Date: May 8, 2025

(54) HETERODIMERIC POLYPEPTIDE COMPLEXES COMPRISING IL-15 VARIANTS AND USES THEREOF

(71) Applicant: WUXI BIOLOGICS IRELAND LIMITED, DUNDALK, CO LOUTH (IE)

(72) Inventors: Yu LIANG, Shanghai (CN); Mengmeng SUN, Shanghai (CN); Lei WU, Shanghai (CN); Ruipu XIN, Shanghai (CN); Jianqing XU, Shanghai (CN); Jijie GU, Shanghai (CN)

(21) Appl. No.: 18/835,695

(22) PCT Filed: Feb. 28, 2023
(86) PCT No.: PCT/CN2023/078743

§ 371 Date: Aug. 2, 2024

(30) Foreign Application Priority Data
Feb. 28, 2022 (WO) PCT/CN2022/078326

(51) Int. Cl. C07K 14/54; A61K 38/00; A61K 45/06; A61K 47/64; A61P 35/00; C07K 14/715; C07K 16/28; C07K 19/00

(52) U.S. Cl. CPC C07K 14/5443; A61K 45/06; A61K 47/64; A61P 35/00; C07K 14/7155; C07K 16/2818; C07K 19/00; A61K 38/00; C07K 2317/569; C07K 2319/00

(57) ABSTRACT

Provided in the present disclosure are IL-15 variants, IL-15/IL-15Rα-Fc fusion proteins comprising the IL-15 variants and IL-15/IL-15Rα fusion protein comprising the IL-15 variants the methods of producing the same and the uses thereof. The IL-15 variants, fusion proteins, and polypeptide complexes of the disclosure can be used as a potent agent for the treatment of cancers and infectious diseases.

### CROSS REFERENCE

This application claims priority to International Patent Application No. PCT/CN2022/078326, filed on Feb. 28, 2022, the entire contents of which is incorporated herein by reference.

### FIELD

This application generally relates to IL-15 proteins and polypeptide complexes comprising the IL-15 proteins. More specifically, the application relates to IL-15 variants, fusion proteins, heterodimeric IL-15/IL-15Rα-Fc polypeptide complexes, a method for preparing the same, and the uses thereof.

### BACKGROUND

Recombinant pro-inflammatory cytokines (IL-2, IFNα and TNFα, etc.) have been approved for cancer treatment for decades, their efficacies are often limited mainly due to short half-life and toxicity. IL-15, an IL-2 family proinflammatory cytokine important for anti-viral and anti-tumoral immunity, is involved in the proliferation and survival of many immune cells (especially the cytolytic T/NK cell), without induction of immuno-suppressive Treg and activation-induced cell death in T/NK cells. IL-15 based therapies could boost T/NK cells to benefit anti-tumor immunity and enhance the therapeutic efficacy of current immunotherapies.

IL-15 is produced in monocytes and dendritic cells and is primarily presented as a membrane-bound heterodimeric complex with IL-15 receptor α (IL-15Rα) present in the same cells. Its effects are realized through trans-presentation of the IL-15/IL-15Rα complex to NK cells and T cells expressing IL-2Rβ and common gamma chain.

IL-15 and its variants have been tested in various clinical trials. However, its potential in cancer immunotherapy is still greatly limited by its immune-toxicity and short half-life. There remains a need to develop novel IL-15 variants, conjugates, fusion proteins, and polypeptide complexes to improve treatment efficacy.

The present disclosure provides IL-15 variants and corresponding heterodimeric polypeptide complexes which have reduced toxicity and enhanced PK/PD profile. These peptides and complexes may serve as a novel immunotherapy agent with improved anti-tumor efficacy, especially in combination with other treatment options.

### SUMMARY

These and other objectives are provided for by the present disclosure which, in a broad sense, is directed to compounds, methods, compositions and articles of manufacture that provide proteins with improved efficacy. The benefits provided by the present disclosure are broadly applicable in the field of therapeutics and diagnostics and may be used in conjunction with antibodies that react with a variety of targets.

In some aspects, the present disclosure provides a IL-15 variant peptide, wherein the IL-15 variant peptide has a lower potency in stimulating immune cell proliferation (such as NK and/or CD8+ T cell proliferation) compared to wild-type IL-15, and comprises an amino acid sequence with a truncation of 1-30, e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids from the N terminal, the C terminal or both the N and C terminals compared to SEQ ID No: 1.

In some embodiments, the IL-15 variant peptide is consisted of a truncate of SEQ ID No: 1, which is truncated by 1-12 amino acids form the C terminal, the N terminal or both the N and C terminals. In some embodiments, the IL-15 variant peptide is truncated by 1-12 amino acids (more preferably 1-5 amino acids) form the C terminal of SEQ ID No: 1. In some embodiments, the IL-15 variant peptide is truncated by 1-12 amino acids form the N terminal of SEQ ID No: 1. In some embodiments, the IL-15 variant peptide is consisted of a truncation of SEQ ID No: 1 truncated from both the C and N terminals and the number of total truncated amino acids is 2-12.

In some embodiments, the IL-15 variant peptide comprises the amino acid sequence of any of SEQ ID Nos: 51-69. For example, the IL-15 variant peptide may be consisted of the amino acid sequence of SEQ ID No: 51.

In some embodiments, the IL-15 variant peptide has a lower potency (i.e. retains the stimulating capability but at a reduced level) in stimulating the proliferation and activation of immune cells, such as NK cells, DC cells and T cells. The immune cells are not limited to human immune cells and can also be derived from other species such as mouse, rat and cynomolgus monkey.

In some embodiments, the potency of the IL-15 variant peptide as disclosed herein in stimulating NK and/or CD8+ T cell proliferation is reduced by more than 10 folds, more than 50 folds, more than 100 folds, more than 200 folds, more than 300 folds, more than 400 folds, more than 500 folds, more than 600 folds, more than 700 folds, more than 800 folds, more than 900 folds, more than 1000 folds, more than 1500 folds, more than 2000 folds or even more compared to wild-type IL-15, as measured in proliferation and activation assays by FACS. The proliferation and activation assays may use Ki-67 and/or pSTAT5, among others, as a marker or indicator.

In some aspects, the present disclosure provides a fusion protein comprising the IL-15 variant as disclosed herein operably linked to a non-IL-15 moiety. The non-IL-15 moiety can be a moiety that enhances the half-life of the fusion protein in vivo, such as a moiety selected from PEGs, lipids, Fc region, human serum albumin (HSA) and anti-HSA moiety. In some embodiments, the non-IL-15 moiety is an Fc region such as a human wild-type IgG1 Fc, IgG2 Fc, IgG3 Fc, IgG4 Fc, or a variant thereof. The non-IL-15 moiety can also be an IL-15Rα domain. In some embodiments, the IL-15Rα domain comprises or consists of a wild-type IL-15Rα protein, an IL-15Rα variant or any fragment thereof that retains IL-15 binding activity, such as a fragment as set forth in SEQ ID No: 2.

In some embodiments, the IL-15 variant peptide is directly linked to the non-IL-15 moiety, or indirectly linked to the non-IL-15 moiety via a linker such as a peptide linker, including a GS linker such as (G4S)n with n=1-10 (i.e. 1, 2, 3, 4, 5, 6, 7, 8, 9 or 10).

In some aspects, the present disclosure provides a polypeptide complex comprising an IL-15 variant domain, an IL-15Rα domain, a first dimerization domain and a second dimerization domain, wherein:
the IL-15 variant domain comprises an IL-15 variant peptide as described above, i.e. having a lower potency in stimulating proliferation or activation of immune cells (such as NK and/or CD8+ T cells) compared to wild-type IL-15, and comprising an amino acid sequence truncated by 1-30 (e.g. 1-12) amino acids from the C terminal, the N terminal, or both the N and C terminals compared to SEQ ID No: 1;
the first dimerization domain and the second dimerization domain associates together to form a dimer.

In some embodiments, the polypeptide complex as disclosed herein has a lower potency in stimulating proliferation or activation of immune cells (such as NK and/or CD8+ T cells) compared to a corresponding polypeptide complex comprising wild-type IL-15 instead of the IL-15 variant.

In some embodiments, the IL-15 variant peptide comprised in the polypeptide complex comprises the amino acid sequence of any of SEQ ID Nos: 51-69, especially SEQ ID No: 51.

In some embodiments, the IL-15Rα domain of the polypeptide complex comprises or consists of a wild-type IL-15Rα protein, an IL-15Rα variant or any fragment thereof that retains IL-15 binding activity, such as a fragment as set forth in SEQ ID No: 2. the IL-15Rα domain may be on the same chain as the IL-15 variant domain or on separate chains.

In some embodiments, the polypeptide complex comprises two chains, wherein, from N terminal to C terminal:
the first chain comprises the IL-15 variant domain operably linked to the first dimerization domain; and
the second chain comprises the IL-15Rα domain operably linked to the second dimerization domain.

In some embodiments, the polypeptide complex comprises one or more targeting moieties such as an antigen-binding moiety. The antigen-binding moiety may be located at the N terminal or C terminal of the dimerization domain. The antigen-binding moiety may adopt a VHH, Fab, scFv (single chain variable fragment), VH or VL format.

In some embodiments, the polypeptide complex comprises two antigen-binding moieties, wherein the first antigen-binding moiety is operably linked to the first dimerization domain, and the second antigen-binding moiety is operably linked to the second dimerization domain.

The antigen-binding moiety as disclosed herein may specifically bind to a wide variety of antigens, including but not limited to, PD-1, PD-L1, PD-L2, CTLA-4, LAG3, TIM-3, TIM4, 4-1BB, OX-40, OX-40L, GITR, A2aR, TIGIT, CD96, PVRIG, CD226, 5T4, VISTA, VSIG3, VSIG4, ICOS, CD28, CD3, CD4, CD8, CD45, CD44v6, CD27, CD47, SIRPAα, SLAMF7, CD24, Siglec10, Siglec15, Siglec8, VSIR, VSIG4, PSGL-1, C5AR1, BTN1A1, BTN3A1, CD70, RANKL, CSF1R, CSF2RB, TNFRSF1/1a/1b, BDCA2, BTLA, C5aR, NKG2A, NKG2D, NKp30, NKp46, CD16a, CD56, CD166, FCGR3, CD2, Neurophilin-1, CCR8, CCR2, CCR4, CCR5, CCR6, CCR7, CCR8, GCGR, CXCR2, CXCR4, CXCR5, CALCRL, ETAR, GLP1R, CX3CR1, GPR1, GPR17, GPR20, GPR30, GPR34, GPR-65, GPCR78, GPRC5D, GPR84, LGR4, LGR5, VEGF, VEGFR, HER2, HER3, Trop2, pCAD, ERα, EGFR, de2-7 EGFR, EGFRvIII, PSMA, PSCA, PSA, TAG-72, SEZ6, SEZ6L, SEZ6L2, SEMA4D, DLL3, GD2, GPC3, KLB, KLRB1, KLRG1, GPC1, PCSK9, EpCAM, p-Cadherin, Caludin 6, Caludin 18.2, FGFR2b, FGFR3, FGFR4, MUC1, MUC13, MUC16, MUC17, MUCL3, FolRa, TfR, TF, TFR, TFPI, c-Met, NY-ESO-1, GUCY2C, LIV-1, Integrin αvβ6, Integrin α10β1, Integrin α3, Integrin α5β4, Integrin αvβ3, Integrin αvβ8, ROR1, ROR2, PRLR, PTK7, B7-H3, Nectin-4, NetG1, Ax1, CD147, LRRC15, Napi2b, STEAP1, LY6G6D, LYPD1, MACRO, MerTK, MICA, MICB, MSLN, Mkars, G12D, CDH3, CDH6, CDH17, APLA2, CAIX, CD46, CD47, CLDN6, EphA3, Fucosyl-GM1, ITGA3, Kallikrein, MISRII, Podocalyxin, RON, ROBO1, PAUF, PLA2, Podocalyxin, PRLR, PTK7, TM4SF1, TMEFF2, TREAKR, TREM-1, TREM-2, uPARAP, TYRP1, KAAG1, RU2AS, CD146, CD63, Endoglin, Globo H, IGF-IR, TEM1, TEM8, TAX1BP3, ADAM-9, ENPP3, EphA2, EphA3, FcRH5, NaPi3b, TWEAK, DLK1, SORT1, SSTR2, STEAP1, CD25, CD39, GARP, LRRC33, LAIR1, LAMP3, LAP, LEPR, LILRB1, LILRB2, LILRB4, RAGE, FGL1, TPBG, PDGFRB, TGFBR2, CEACAM1, CEACAM5, CEACAM6, Carcinoembryonic antigen (CEA), ICAM1, A33, CAMPATH-1 (CDw52), Carboanhydrase IX (MN/CA IX), CD248, PDPN, ITGB1, ITGAV, CD20, CD19, CD21, CD22, CLL, BCMA, DCLK1, DDR1, DLK1, DPEP3, DKK1, CD5, CD13, CD30, CD33, CD34, CD36, CD37, CD38, CD43, CD52, CD55, CD94, CD99, CD7, CD71, CD73, CD74, CD79a, CD79b, CD229, CD132, CD133, G250, CSF1R (CD115), HLA-DR, HLA-G, HTRA1, TRA-1-60, IGFR, IL-2 receptor, MCSP (Melanoma-associated cell surface chondroitin sulphate proteoglycane), ART1, ASGR1, B7H3, B7-H4, B7H6, CD124, c-Kit (CD117), CD7, Clex12A, Clever-1, IL-13RA2, IL-11RA, IL-31RA, IL-4RA, IFNAR, ActRIIb, IL-7R, SLAMF7, Fms-like tyrosine kinase 3 (FLT-3, CD135), GFRA1, BTLA, GloboH, CSF2RB, chondroitin sulfate proteoglycan 4 (CSPG4, melanoma-associated chondroitin sulfate proteoglycan), ITGA4, Clec5a, Clec7a, Clec9a, Clec12a, CLEC14, CD205, CD206, CD200R1, CD228, CD229, CD40, CD40L, FcRn, TLR8, TLR9, TNFR2, LTBR, CD44, CD93, PDGF, PDGFR-alpha (CD140a), PDGFR-beta (CD140b), CD146, CD147, CRTH2, TNF-α, TGF-β, IL1RAcP, TSLP, DR5, ST2, fibroblast activating protein (FAP), CDCP1, Derlin1, Tenascin, frizzled 1-10, the vascular antigens VEGFR2 (KDR/FLK1), VEGFR3 (FLT4, CD309), Endoglin, Tie2 and other TAAs, I/O checkpoints, tumor microenvironment targets, or autoimmune and inflammatory diseases associated targets.

In some embodiments, the first and second antigen-binding moieties are in VHH format and specifically bind to PD-1. Preferably, the first and second antigen-binding moieties have a same amino acid sequence. In some embodiments, the first and second antigen-binding moieties comprise the amino acid sequence as set forth in SEQ ID No: 80.

The term operably linked as mentioned herein includes a direct linkage and a linkage via a linker, such as a peptide linker. The peptide linker may be a partial or complete IgG hinge region. The peptide linker may also be a conventionally used peptide linker such as a GS series linker, including (GS)n, (G2S)n, (G3S)n, (G4S)n linker, etc. In some embodiments, the dimerization domain is operably linked to the antigen-binding moiety via a (G4S)n linker with n=1-10.

In some embodiments, the first dimerization domain is one chain of an immunoglobulin Fc region, and the second dimerization domain is the other chain of the immunoglobulin Fc region. The Fc region comprises a human IgG Fc such as a human IgG1 Fc, IgG2 Fc, IgG3 Fc, IgG4 Fc, or a variant thereof.

In some embodiments, the Fc variant comprises one or more substitutions compared to wild type human Fc to increase the stability of the Fc region, promote heterodimerization, and/or alter the binding to Fc receptors.

In some embodiments, the Fc variant comprises:
(a) a human IgG4 Fc engineered to comprise one or more of the following: S228P mutation, F234A/L235A mutations, M252Y/S254T/T256E mutations and “knob into hole” structure; or
(b) a human IgG1 Fc engineered to comprise one or more of the following: M252Y/S254T/T256E mutations, G236R/L328R mutations and “knob into hole” structure.

In some embodiments, the Fc variant comprises:
(a) an amino acid sequence with at least 80% identity to SEQ ID No: 3 for one chain, and an amino acid sequence with at least 80% identity to SEQ ID No: 4 for the other chain;
(b) an amino acid sequence with at least 80% identity to SEQ ID No: 5 for one chain, and an amino acid sequence with at least 80% identity to SEQ ID No: 6 for the other chain; or
(c) an amino acid sequence with at least 80% identity to SEQ ID No: 7 for one chain, and an amino acid sequence with at least 80% identity to SEQ ID No: 8 for the other chain.

In some embodiments, the first chain of the polypeptide complex comprises an amino acid sequence as set forth in SEQ ID No: 13, 15, 17, 19, 21, 23, 25, 27, 29, 31, 33, 35, 37, 39, 41, 43, 45, 47, 49, 70, 72, 74 or 76, and/or the second chain of the polypeptide complex comprises an amino acid sequence as set forth in SEQ ID No: 14, 16, 18, 20, 22, 24, 26, 28, 30, 32, 34, 36, 38, 40, 42, 46, 48, 50, 71, 73, 75 or 77.

In some embodiments, the polypeptide complex comprises two chains, wherein:
(a) the first chain comprises an amino acid sequence as set forth in SEQ ID No: 19, and the second chain comprises an amino acid sequence as set forth in SEQ ID No: 20;
(b) the first chain comprises an amino acid sequence as set forth in SEQ ID No: 70, and the second chain comprises an amino acid sequence as set forth in SEQ ID No: 71;
(c) the first chain comprises an amino acid sequence as set forth in SEQ ID No: 72, and the second chain comprises an amino acid sequence as set forth in SEQ ID No: 73;
(d) the first chain comprises an amino acid sequence as set forth in SEQ ID No: 74, and the second chain comprises an amino acid sequence as set forth in SEQ ID No: 75;
(e) the first chain comprises an amino acid sequence as set forth in SEQ ID No: 21, and the second chain comprises an amino acid sequence as set forth in SEQ ID No: 22;
(f) the first chain comprises an amino acid sequence as set forth in SEQ ID No: 23, and the second chain comprises an amino acid sequence as set forth in SEQ ID No: 24;
(g) the first chain comprises an amino acid sequence as set forth in SEQ ID No: 25, and the second chain comprises an amino acid sequence as set forth in SEQ ID No: 26; or
(h) the first chain comprises an amino acid sequence as set forth in SEQ ID No: 76, and the second chain comprises an amino acid sequence as set forth in SEQ ID No: 77.

In some aspects, the present disclosure provides a nucleic acid molecule, comprising a nucleic acid sequence encoding the first and/or second chain of the polypeptide complex, the fusion protein, or the IL-15 variant peptide as disclosed herein.

In some aspects, the present disclosure provides a vector comprising the nucleic acid molecule as disclosed herein. In some aspects, the present disclosure provides a host cell comprising the vector or the nucleic acid molecule as disclosed herein.

In some aspects, the present disclosure provides a pharmaceutical composition comprising the polypeptide complex, the fusion protein or the nucleic acid molecule as disclosed herein, as well as a pharmaceutically acceptable carrier.

In some aspects, the present disclosure provides an immunoconjugate comprising the polypeptide complex or the fusion protein as disclosed herein conjugated to a moiety selected from a chemical compound (such as a cytotoxic agent, a cytostatic agent), polypeptide, carbohydrate, lipid, nucleic acid or a combination thereof.

In some aspects, the present disclosure provides a method for producing the polypeptide complex or the fusion protein as disclosed herein, comprising the steps of:
culturing a host cell comprising a vector(s) encoding one or both chains of the polypeptide complex or the fusion protein; and
isolating the polypeptide complex or the fusion protein from the host cell culture.

In some aspects, the present disclosure provides a method of modulating an immune response in a subject, comprising administering the polypeptide complex or the pharmaceutical composition as disclosed herein to the subject, optionally the immune response is NK cell or T cell (such as CD8+ T cell) related.

In some aspects, the present disclosure provides a method for treating or preventing cancer, an infectious disease, an immunodeficiency or lymphopenia in a subject, comprising administering an effective amount of the polypeptide complex or the pharmaceutical composition as disclosed herein to the subject.

In some embodiments, the method further comprises administering an additional anti-tumor therapy, such as cellular immunotherapy including tumor-infiltrating lymphocyte (TIL) therapy, T cell receptor T cell (TCR-T) therapy, chimeric antigen receptor (CAR) T cell therapy, NK cell therapy, oncolytic virus therapy, targeted therapy, chemotherapy and gene therapy. The administration of the additional anti-tumor therapy may be performed before, after or simultaneously with the administration of the polypeptide complex or the pharmaceutical composition as disclosed herein.

In some aspects, the present disclosure provides a method for treating or preventing cancer, an infectious disease, an immunodeficiency or lymphopenia in a subject, comprising administering a cell therapy or a gene therapy, wherein the cell therapy and the gene therapy comprises delivering the nucleic acid molecule as disclosed herein to the subject.

In some embodiments, the cell therapy is selected from tumor-infiltrating lymphocyte (TIL) therapy, T cell receptor (TCR) therapy, chimeric antigen receptor (CAR) T cell therapy, and NK cell therapy. In some embodiments, the gene therapy uses lentivirus, AAV, poxvirus, herpes zoster virus, oncolytic virus, or other RNA/DNA vectors for gene delivery.

In some embodiments, the cancer is selected from breast cancer, lung cancer (such as NSCLC), colon cancer, ovarian cancer, melanoma, bladder cancer, renal cell carcinoma, liver cancer, prostate cancer, stomach cancer, pancreatic cancer, lymphoma (such as non-Hodgkin's lymphoma and diffuse large B-cell lymphoma), leukemia (such as chronic lymphocytic leukemia), and multiple myeloma.

In some embodiments, the infectious disease is caused by a viral, bacterial, fungal or parasite infection.

In some aspects, the present disclosure provides use of the polypeptide complex, the fusion protein or the nucleic acid molecule as disclosed herein in the manufacture of a medicament for preventing, treating and/or managing cancer, an infectious disease, an immunodeficiency or lymphopenia.

In some aspects, the present disclosure provides the polypeptide complex, the fusion protein or the nucleic acid molecule as disclosed herein for use in treating or preventing cancer, an infectious disease, an immunodeficiency or lymphopenia.

In some aspects, the present disclosure provides a kit, comprising a container comprising the polypeptide complex, the fusion protein or the nucleic acid molecule as disclosed herein.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein.

### BRIEF DESCRIPTION OF THE FIGURES

FIG. 1 illustrates the structures of IL-15/Rα fusion protein. (A) a heterodimeric IL-15/Rα-Fc polypeptide complex according to some embodiments of the disclosure. (B) a IL-15/Rα fusion protein according to some embodiments of the disclosure. (C) a heterodimeric IL-15/Rα-Fc polypeptide complex that further comprises an antigen-binding moiety(ies) according to some embodiments of the disclosure.

FIG. 2 illustrates heterodimeric IL-15/Rα-Fc polypeptide complexes promote proliferation of CD8+ T cells (A) and NK cells (B) of human PBMCs. Human PBMCs were cultured with indicated proteins for 4 days. Proliferation of CD8+ T cells (CD3+CD8+) and NK cells (CD3-CD56+) were presented as Ki67+ (%) of CD8+ T cells and Ki67+ (%) of NK cells, respectively.

FIG. 3 illustrates W327151-TXU1.P1-V0013.uIgG4v322 promotes proliferation of cyno CD8+ cells (A) and CD16+ cells (B). Cynomolgus PBMCs were cultured with indicated proteins for 4 days. Proliferation of CD8+ cells and CD16+ cells were presented as Ki67+ (%) of CD8+ cells and Ki67+ (%) of CD16+NK cells, respectively.

FIG. 4 illustrates W327151-TXU1.P1-V0013.uIgG4v322 promotes proliferation of mouse CD8+ T cells (A) and NK cells (B). Mouse spleen cells were cultured with indicated proteins for 4 days. Proliferation of CD8+ T cells (CD3+CD8a+) and NK cells (CD3-CD335+) were presented as Ki67+ (%) of CD8+ T cells and Ki67+ (%) of NK cells, respectively.

FIG. 5 illustrates W327151-TXU1.P1-V0013.uIgG4v322 binding curve on HH cell line.

FIG. 6 illustrates SPR curves showing binding of pre-complexed human IL-2Rβ with heterodimeric IL-15/Rα-Fc polypeptide complexes (1:1) to human IL-2Rγc immobilized on a sensor chip.

FIG. 7 illustrates fusion protein potency on MLR assay. T cell activation was determined by IFN-γ (A) and IL-2 (B) in the supernatant detected by ELISA.

FIG. 8 illustrates the serum concentration profiles of W327151-TXU1.P1-V0013.uIgG4v322 and W327151-BMK1 in C57BL/6 mice, detected by ELISA of Fc-Fc (A) or Fc-IL15 (B).

FIG. 9 illustrates body weight change of maximum tolerance dose (MTD) assay. Mice were treated with IL-15/Rα-Fc polypeptide complexes in different doses.

FIG. 10 illustrates pharmacodynamics effect of murine peripheral lymphocyte, NK, T, CD8+T, CD4+T and Treg cell count upon treatment of W327151-TXU1.P1-V0013.uIgG4V322 or WT IL-15 in different doses.

FIG. 11 illustrates murine peripheral NK and CD8+ T cell proliferation and activation upon treatment of W327151-TXU1.P1-V0013.uIgG4V322 or WT IL-15 in different doses.

FIG. 12 illustrates pharmacodynamics effect of peripheral Tcm, Tem and naïve CD8+ T cell count upon treatment of W327151-TXU1.P1-V0013.uIgG4V322 or WT IL-15 in different doses.

FIG. 13 illustrates albumin concentration (A) and inflammatory cytokine release (B) in mouse plasma after treatment of W327151-TXU1.P1-V0013.uIgG4V322 or WT IL-15 in different doses.

FIG. 14 illustrates the change in tumor volume of CT26 colon carcinoma model after treated with W327151-TXU1.P1-V0013.uIgG4V322 or WT IL-15 in different doses.

FIG. 15 illustrates the change in tumor volume of A375 humanized model after treated with W327151-TXU1.P1-V0013.uIgG4V322, W327151-BMK1 or W327151-BMK2.

FIG. 16 illustrates the change in tumor volume of Daudi CDX model after treated with W327151-TXU1.P1-V0013.uIgG4V322 or WT IL-15 in different doses.

### DETAILED DESCRIPTION

While the present disclosure may be embodied in many different forms, disclosed herein are specific illustrative embodiments thereof that exemplify the principles of the disclosure. It should be emphasized that the present disclosure is not limited to the specific embodiments illustrated. Moreover, any section headings used herein are for organizational purposes only and are not to be construed as limiting the subject matter described.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like. In this application, the use of “or” means “and/or” unless stated otherwise. Furthermore, the use of the term “comprising,” as well as other forms, such as “comprises” and “comprised,” is not limiting. In addition, ranges provided in the specification and appended claims include both end points and all points between the end points.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. See, e.g., Abbas et al., Cellular and Molecular Immunology, 6^{th} ed., W.B. Saunders Company (2010); Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). The nomenclature used in connection with, and the laboratory procedures and techniques of, analytical chemistry, synthetic organic chemistry, and medicinal and pharmaceutical chemistry described herein are those well-known and commonly used in the art.

### Definitions

In order to better understand the disclosure, the definitions and explanations of the relevant terms are provided as follows.

The term “IL-15”, as used herein, refers to Interleukin-15 and is intended to encompass any forms of IL-15, for example, 1) native unprocessed IL-15 molecule, “full-length” IL-15 protein or naturally occurring variants of IL-15; 2) any form of IL-15 that results from processing in the cell; or 3) full length or a modified form. A wild-type IL-15 is a 14-15 kDa member of the four-α-helix bundle family of cytokines that is involved in natural killer (NK) cell differentiation, T-cell functions, and the host response to intracellular pathogens. An example of the amino acid sequence of human wild-type IL-15 is shown in SEQ ID No: 1. As used in the disclosure, the term “IL-15” or “IL-15 domain” includes both wild-type IL-15 and IL-15 variants.

The term “IL-15Rα” or “IL-15 receptor α”, as used herein, refers to a high-affinity receptor of IL-15, which binds to IL-15 and transduces signals in the presence of the IL-15Rβ and γc (common γ chain). The full-length human IL-15Rα is a type-1 transmembrane protein with a signal peptide, an extracellular domain, a transmembrane domain and a cytoplasmic tail. As used in the disclosure, the term “IL-15Rα” or “IL-15Rα domain” includes both wild-type IL-15Rα and IL-15Rα variants and fragments thereof. As described herein and known in the art, the IL-15Rα protein contains a “sushi domain”, which is the shortest region of the receptor that retains IL-15 binding activity (as shown in SEQ ID No: 2). In some embodiments, the “sushi domain”, i.e. the peptide as shown in SEQ ID No: 2, is used to construct the IL-15/Rα-Fc polypeptide complexes as disclosed herein.

The term “variant”, with regard to polypeptide or protein, means a biologically active polypeptide which includes one or more amino acid mutations in the native protein sequence. Optionally, the one or more amino acid mutations include amino acid substitution, insertion and deletion at certain positions within the amino acid sequence. A variant has preferably at least about 80%, more preferably at least about 90%, and more preferably at least about 95% amino acid sequence identity with the corresponding native sequence polypeptide. Such variants include, for instance, polypeptides wherein one or more amino acid residues are truncated at the N- and/or C-terminus of the polypeptide. Variants thereof for use in the disclosure can be prepared by a variety of methods well known in the art, such as synthesizing or recombinantly generating the DNA encoding the variant, and thereafter expressing the DNA in cell culture. In certain embodiments as disclosed herein, an IL-15 variant is truncated by one or more amino acid residues at the N terminal and/or C terminal compared to the wild-type IL-15 protein.

The term “Fc”, as used herein, has a same meaning as used with regard to an antibody, which refers to that portion of the antibody comprising the second (CH2) and third (CH3) constant regions of a first heavy chain bound to the second and third constant regions of a second heavy chain via disulfide bonding. The Fc region may also comprise part or whole of the hinge region. The Fc region of the antibody is responsible for various effector functions such as ADCC and CDC, but does not function in antigen binding. In the present disclosure, the term “Fc” includes both wild-type Fc and Fc variants.

The term “modification”, with respect to an amino acid residue/position as used herein, refers to a change of a primary amino acid sequence as compared to a starting amino acid sequence, wherein the change results from a sequence alteration involving said amino acid residue/positions. For example, typical modifications include substitution of the residue (or at said position) with another amino acid (e.g., a conservative or non-conservative substitution), and insertion of one or more amino acids adjacent to said residue/position. An “amino acid substitution”, or variation thereof, refers to the replacement of an existing amino acid residue in a predetermined (starting) amino acid sequence with a different amino acid residue. Generally, the modification results in alteration in at least one physicobiochemical activity of the variant polypeptide compared to a polypeptide comprising the starting (or “wild type”) amino acid sequence. For example, in an IL-15 variant, a physicobiochemical activity that is altered can be binding affinity, binding capability and/or binding effect upon a target molecule. As used herein, two or more substitutions in an amino acid sequence may be expressed with “+” or “/” between each substitution.

The term “fusion protein”, as used herein, refers to a polypeptide having two (or more) portions operably linked together, where each of the portions is a polypeptide having a different property. The property may be a biological property, such as activity in vitro or in vivo. The property may also be a simple chemical or physical property, catalysis of a reaction, etc. The two portions may be linked directly via a single peptide bond or indirectly via a linker, such as a peptide linker containing one or more amino acid residues. Generally, the two portions and the peptide linker will be in reading frame with each other. In certain embodiments, the two portions of the fusion protein are IL-15 protein domain and Fc domain, or IL-15Rα protein domain and Fc domain, or IL-15 protein domain and IL-15Rα protein domain. The two fusion proteins may be associated together to form a heterodimer. In a broad sense, when mentioning “fusion protein” in the present specification, depending on the context, it may refer to a single chain IL-15-comprising fusion protein or a heterodimeric polypeptide complex as defined below.

As used herein, the terms “heterodimer”, “heterodimeric fusion protein”, “heterodimeric IL-15 polypeptide complex” and “IL-15/Rα-Fc polypeptide complex” may be used interchangeably, and refer to a heterodimer protein comprising two chains, each chain comprising a dimerization domain that can be associated together to form a dimer, and the IL-15 domain may be on the same or other chain with the IL-15Rα domain. In some embodiments, the first chain is a fusion protein of IL-15 protein domain and Fc domain, the second chain is a fusion protein of IL-15Rα protein domain and Fc domain. Such heterodimeric fusion proteins are structurally similar to an antibody, in view that the Fab part in an antibody is replaced by an IL-15/IL-15Rα complex formed by interaction between the IL-15 and IL-15Rα domains. Furthermore, the polypeptide complex may be fused to one or more antigen-binding moiety(ies) or antigen-binding fragments derived from an antibody. Such polypeptide complexes combine the immune cell proliferation and activation function of the IL-15/IL-15Rα portion and the site-directed targeting of the antigen-binding moiety.

The terms “operably linked” refer to a juxtaposition, with or without a spacer or linker, of two or more biological sequences of interest in such a way that they are in a relationship permitting them to function in an intended manner. When used with respect to polypeptides, it is intended to mean that the polypeptide sequences are linked in such a way that permits the linked product to have the intended biological function, whether retaining the respective functions of each of the polypeptide sequence or leading to a reduced function of one of the polypeptide sequence via steric hinderance imposed by other polypeptide sequence(s). For example, an antibody variable region may be operably linked to a constant region so as to provide for a stable product with antigen-binding activity; an IL-15 domain may be operably linked (directly or indirectly via a linker) to the Fc domain. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide.

The term “gene therapy”, as used herein, refers to a therapy that aims to treat diseases by replacing, inactivating or introducing genes into cells either inside the body (in vivo) or outside of the body (ex vivo). In some embodiments, a DNA or RNA sequence encoding the IL-15 variant(s) and fusion proteins as disclosed herein is administered to a subject. Gene therapy includes using lentivirus, AAV, poxvirus, herpes zoster virus, oncolytic virus and other RNA/DNA vectors to deliver the DNA or RNAs encoding the heterodimeric polypeptide complex or the pharmaceutical composition as disclosed herein.

The term “cell therapy”, as used herein, refers to a therapy that aims to treat diseases by restoring or altering certain sets of cells or by using cells to carry a therapy through the body. With cell therapy, cells are cultivated or modified outside the body before being injected into the patient. The cells may originate from the patient (autologous cells) or a donor (allogeneic cells). Current cell therapy includes using CAR-T, TCR-T, TIL, CAR-NK, CAR-γδT, CAR-macrophage and other engineered immune cells. In some embodiments herein, the treatment method further includes the cell therapy.

The term “EC₅₀,” as used herein, which is also termed as “half maximal effective concentration” refers to the concentration of a drug, antibody or toxicant which induces a response halfway between the baseline and maximum after a specified exposure time. In the context of the application, EC₅₀ is expressed in the unit of “nM”.

The term “isolated,” as used herein, refers to a state obtained from natural state by artificial means. If a certain “isolated” substance or component is present in nature, it is possible because its natural environment changes, or the substance is isolated from natural environment, or both. For example, a certain un-isolated polynucleotide or polypeptide naturally exists in a certain living animal body, and the same polynucleotide or polypeptide with a high purity isolated from such a natural state is called isolated polynucleotide or polypeptide. The term “isolated” excludes neither the mixed artificial or synthesized substance nor other impure substances that do not affect the activity of the isolated substance.

The term “vector,” as used herein, refers to a nucleic acid vehicle which can have a polynucleotide inserted therein. When the vector allows for the expression of the protein encoded by the polynucleotide inserted therein, the vector is called an expression vector. The vector can have the carried genetic material elements expressed in a host cell by transformation, transduction, or transfection into the host cell. Vectors are well known by a person skilled in the art, including, but not limited to plasmids, phages, cosmids, artificial chromosome such as yeast artificial chromosome (YAC), bacterial artificial chromosome (BAC) or P1-derived artificial chromosome (PAC); phage such as λ phage or M13 phage and animal virus. The animal viruses that can be used as vectors, include, but are not limited to, retrovirus (including lentivirus), adenovirus, adeno-associated virus, herpes virus (such as herpes simplex virus), pox virus, baculovirus, papillomavirus, papova virus (such as SV40). A vector may comprise multiple elements for controlling expression, including, but not limited to, a promoter sequence, a transcription initiation sequence, an enhancer sequence, a selection element and a reporter gene. In addition, a vector may comprise origin of replication.

The term “host cell,” as used herein, refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; or human tissues or hybridoma cells, yeast cells, and insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell”.

The term “identity,” as used herein, refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al, 1988, SIAMJ. Applied Math. 48:1073.

The term “transfection,” as used herein, refers to the process by which nucleic acids are introduced into eukaryotic cells, particularly mammalian cells. Protocols and techniques for transfection include but not limited to lipid transfection and chemical and physical methods such as electroporation. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al, 1981, Gene 13:197. In a specific embodiment of the disclosure, human IL-15 gene was transfected into 293F cells.

The term “fluorescence-activated cell sorting” or “FACS,” as used herein, refers to a specialized type of flow cytometry. It provides a method for sorting a heterogeneous mixture of biological cells into two or more containers, one cell at a time, based upon the specific light scattering and fluorescent characteristics of each cell (FlowMetric. “Sorting Out Fluorescence Activated Cell Sorting”. Retrieved 2017-11-09). Instruments for carrying out FACS are known to those of skill in the art and are commercially available to the public. Examples of such instruments include FACS Star Plus, FACScan and FACSort instruments from Becton Dickinson (Foster City, Calif.) Epics C from Coulter Epics Division (Hialeah, Fla.) and MoFlo from Cytomation (Colorado Springs, Colo.).

The term “subject” includes any human or nonhuman animal, preferably mammals, more preferably humans.

The term “cancer,” as used herein, refers to any or a tumor or a malignant cell growth, proliferation or metastasis-mediated, solid tumors and non-solid tumors such as leukemia and initiate a medical condition.

The term “treatment,” “treating” or “treated,” as used herein in the context of treating a condition, pertains generally to treatment and therapy, whether of a human or an animal, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, preventing or delaying the development of a tumor, or some combination thereof.

The term “an effective amount,” as used herein, pertains to that amount of an active compound, or a material, composition or dosage form comprising an active compound, which is effective for producing some desired therapeutic effect, commensurate with a reasonable benefit/risk ratio, when administered in accordance with a desired treatment regimen. For instance, the “an effective amount,” when used in connection with treatment of diseases or conditions such as cancers, refers to an active agent, a drug or an antibody or antigen-binding portion thereof in an amount or concentration effective to treat the said diseases or conditions.

The term “prevent,” “prevention” or “preventing,” as used herein, with reference to a certain disease condition in a mammal, refers to preventing or delaying the onset of the disease, or preventing the manifestation of clinical or subclinical symptoms thereof.

The term “pharmaceutically acceptable,” as used herein, means that the vehicle, diluent, excipient and/or salts thereof, are chemically and/or physically is compatible with other ingredients in the formulation, and the physiologically compatible with the recipient.

As used herein, the term “a pharmaceutically acceptable carrier and/or excipient” refers to a carrier and/or excipient pharmacologically and/or physiologically compatible with a subject and an active agent, which is well known in the art (see, e.g., Remington's Pharmaceutical Sciences. Edited by Gennaro AR, 19th ed. Pennsylvania: Mack Publishing Company, 1995), and includes, but is not limited to pH adjuster, surfactant, adjuvant and ionic strength enhancer. For example, the pH adjuster includes, but is not limited to, phosphate buffer; the surfactant includes, but is not limited to, cationic, anionic, or non-ionic surfactant, e.g., Tween-80; the ionic strength enhancer includes, but is not limited to, sodium chloride.

As used herein, the term “adjuvant” refers to a non-specific immunopotentiator, which can enhance immune response to an antigen or change the type of immune response in an organism when it is delivered together with the antigen to the organism or is delivered to the organism in advance. There are a variety of adjuvants, including, but not limited to, aluminium adjuvants (for example, aluminum hydroxide), Freund's adjuvants (for example, Freund's complete adjuvant and Freund's incomplete adjuvant), coryne bacterium parvum, lipopolysaccharide, cytokines, and the like. Freund's adjuvant is the most commonly used adjuvant in animal experiments now. Aluminum hydroxide adjuvant is more commonly used in clinical trials.

### IL-15, IL-15Rα Protein Domains and IL-15/IL-15Rα Complex

In some aspects, the present disclosure provides IL-15 variant peptides which comprise one or more modification(s), e.g. truncation from the N-terminal or C-terminal, compared to the wild-type IL-15 protein, such as human wild-type IL-15 protein. The present disclosure further provides IL-15-Fc fusion proteins comprising IL-15 protein domain fused to a Fc domain. The IL-15 protein domain may comprise a wild-type IL-15 protein as set forth in SEQ ID No: 1, or an IL-15 variant comprising one or more deletion(s) compared to the wild-type IL-15 protein.

Residues are designated herein by the one letter amino acid code followed by the IL-15 amino acid position, e.g., W2 is the Trp residue at position 2 of SEQ ID NO: 1, which is the 2^{nd} residue from the N terminal. F110 is the Phe residue at position 110 of SEQ ID NO: 1, which is the 5^{th} residue from the C terminal. Substitutions are designated herein by the one letter amino acid code followed by the IL-15 amino acid position followed by the substituting one letter amino acid code, e.g., D30N is a substitution of the Asp residue at position 30 of SEQ ID NO: 1 with Asn residue.

In some embodiments, the IL-15 variant has a 1 amino acid, 2 amino acid, 3 amino acid, 4 amino acid, 5 amino acid, 6 amino acid, 7 amino acid, 8 amino acid, 9 amino acid, or 10 amino acid truncation, or a truncation of more amino acids from the C terminal compared to SEQ ID No: 1.

In some embodiments, the IL-15 variant has a 1 amino acid, 2 amino acid, 3 amino acid, 4 amino acid, 5 amino acid, 6 amino acid, 7 amino acid, 8 amino acid, 9 amino acid, 10 amino acid truncation, 11 amino acid truncation, 12 amino acid truncation, or a truncation of more amino acids from the N terminal compared to SEQ ID No: 1.

In some embodiments, the IL-15 variant has a truncation of 1, 2, 3, 4, 5, 7, 8, 9, 10 or more amino acids from the C terminal, and a truncation of 1, 2, 3, 4, 5, 6, 7, 8, 9, 10, 11, 12 or more amino acids from the N terminal, compared to SEQ ID No: 1. Specifically, the IL-15 variant may comprise or consist of any of SEQ ID Nos: 51-69.

The truncation of amino acid residues at N and/or C terminal may reduce the interaction interface between IL-15 and IL-2Rβ/γ, for example by disrupting the hydrogen bonding, salt bridge, and/or van der Waals interaction at the interface. By deleting the terminal amino acid residues, the IL-15 variants as disclosed herein provide a reduced binding affinity to IL-15Rβ/γ chain, resulting in reduced (but still existent) potency in stimulating immune cells and thus reduced toxicity in vivo.

In one aspect, provided herein is an IL-15/IL-15Rα complex comprising IL-15 domain associated with IL-15Rα domain. The IL-15 domain may be on different chains with the IL-15Rα domain, or may be operably linked to IL-15Rα domain in the same chain. The IL-15Rα protein domain may comprise a wild-type IL-15Rα protein, an IL-15Rα variant or any fragment thereof that retains the IL-15 binding activity. In some embodiments, the IL-15Rα protein domain comprises IL-15 binding fragment thereof as set forth in SEQ ID No: 2. In some embodiments, the IL-15Rα variant has one or more modification(s), e.g. insertion, substitution and/or deletion, compared to SEQ ID No: 2.

The IL-15 protein domain may associate with the IL-15Rα protein domain in different ways to form an IL-15R/IL-15Rα complex. In some embodiments, the IL-15 protein domain and the IL-15Rα protein domain are self-assembled through ligand-ligand interaction, i.e. the binding affinity between the two domains. In some other embodiments, the two domains are covalently linked via a linker, preferably a peptide linker. In some further embodiments, both domains are engineered to include a cysteine amino acid(s) such that a disulfide bond(s) is/are formed between the two domains.

### IL-15-Fc Fusion Proteins and Heterodimeric IL-15/IL-15Rα-Fc Polypeptide Complexes

In some aspects, the present disclosure provides single chain IL-15-Fc fusion proteins that comprise IL-15 protein domains operably linked to an Fc domain, and IL-15Rα-Fc fusion proteins that comprise IL-15Rα protein domains operably linked to an Fc domain. Further, the two fusion proteins may heterodimerize to form an IL-15/IL-15Rα-Fc polypeptide complex comprising two chains. Each of the IL-15 domain, IL-15Rα domain and Fc domain constituting the heterodimeric IL-15/IL-15Rα-Fc polypeptide complex can be in wild-type format or corresponding variants, as described above. Although the IL-15/IL-15Rα-Fc polypeptide complex as disclosed herein comprises a Fc domain linked to the IL-15/IL-15Rα domain, it is contemplated that other dimerization domains may also be used. For example, the IL-15 domain may be operably linked to a first dimerization domain, and the IL-15Rα domain may be operably linked to a second dimerization domain, and the first and the second dimerization domains are associated to form a dimer.

As used herein, the term “long acting” means an IL-15 protein is fused to or operably linked to a moiety that is capable of increasing the half-life of IL-15 in vivo. Preferably, the moiety may be an immunoglobulin Fc region that provides a prolonged pharmacokinetics (PK) profile, as shown in e.g. a longer serum half-life, increased Cmax, lower serum clearance and improved drug exposure (AUC), or a pronounced and extended pharmacodynamics (PD) effect on lymphocyte expansion, especially proliferation of NK and CD8+ T cells.

In some embodiments, the heterodimeric IL-15/IL-15Rα-Fc polypeptide complexes as disclosed herein comprise two chains, the first chain comprises a fusion of the IL-15 domain with one chain of the Fc domain, the second chain comprises a fusion of the IL-15Rα domain with the other chain of the Fc domain. In some embodiments, the IL-15/IL-15Rα domain is operably linked to the Fc domain. Preferably, the IL-15 or IL-15Rα domain is operably linked to the N terminus of the Fc domain.

In some other embodiments, the IL-15/IL-15Rα-Fc polypeptide complexes as disclosed herein comprise two chains, the first chain comprises a fusion of the IL-15 domain and IL-15Rα domain with one chain of the Fc domain, the second chain comprises the other chain of the Fc domain. For example, the IL-15 domain may be operably linked to the IL-15Rα domain (optionally via a linker), and further operably linked to one chain of the Fc domain, such as IL15-IL15Rα-Fc or IL15Rα-IL15-Fc from N terminal to C terminal, wherein “−” represents an operable linkage.

In some embodiments, the IL-15/IL-15Rα domain is directly linked to the Fc domain, i.e. without a linker. This type of more rigid structure may reduce the potency of IL-15 (wildtype or variant) as desired, possibly due to the structural hindrance caused by Fc domain. Alternatively, the IL-15/IL-15Rα domain is linked to the Fc domain via a linker, such as a peptide linker with one or more amino acid residues. Many suitable linkers are available in the art and can be used here. In some embodiments, the linker may be (G4S)n, (GS)n, (GSGGS)n or (G3S)n, wherein n may be 0-5 (such as 0, 1, 2, 3, 4 or 5).

In some embodiments, the IL-15/IL-15Rα-Fc fusion proteins as disclosed herein comprise two chains, the first chain comprises an IL-15 domain operably linked to one chain of an IgG Fc domain, the second chain comprises a IL-15Rα protein domain operably linked to the other chain of the IgG Fc domain, and the IL-15 domain comprises an IL-15 variant as described above. In some exemplary embodiments, the IL-15 variant has a 1 amino acid, 2 amino acid, 3 amino acid, 4 amino acid, 5 amino acid, 6 amino acid, 7 amino acid, 8 amino acid, 9 amino acid, or 10 amino acid truncation, or a truncation of more amino acids from the C terminal compared to SEQ ID No: 1.

In some exemplary embodiments, the IL-15 variant has a 1 amino acid, 2 amino acid, 3 amino acid, 4 amino acid, 5 amino acid, 6 amino acid, 7 amino acid, 8 amino acid, 9 amino acid, 10 amino acid truncation, 11 amino acid truncation, 12 amino acid truncation, or a truncation of more amino acids from the N terminal compared to SEQ ID No: 1.

In some exemplary embodiments, the IL-15 variant has a truncation of 1, 2, 3, 4, 5, 7, 8, 9, 10 or more amino acids from the C terminal, and a truncation of 1, 2, 3, 4, 5, 6, 7, 8, 9, 10, 11, 12 or more amino acids from the N terminal, compared to SEQ ID No: 1. Specifically, the IL-15 variant may comprise or consist of any of SEQ ID Nos: 51-69.

In some further embodiments, the Fc domain is an IgG1 Fc domain and may comprise SEQ ID No: 3 in one chain and SEQ ID No: 4 in the other chain (referred to as “V805” format). In some further embodiments, the Fc domain is an IgG4 Fc domain and may comprise SEQ ID No: 5 in one chain and SEQ ID No: 6 in the other chain (referred to as “V1” format). In some further embodiments, the Fc domain is an IgG4 Fc domain and may comprise SEQ ID No: 7 in one chain and SEQ ID No: 8 in the other chain (referred to as “V322” format).

For example, in certain embodiments, the IL-15 variant is operably linked to one chain of the Fc domain as set forth in SEQ ID No: 3 while the IL-15Rα protein domain is operably linked to the other chain of the Fc domain as set forth in SEQ ID No: 4; in some other embodiments, the IL-15 variant is operably linked to one chain of the Fc domain as set forth in SEQ ID No: 4 while the IL-15Rα protein domain is operably linked to the other chain of the Fc domain as set forth in SEQ ID No: 3; in certain embodiments, the IL-15 variant is operably linked to one chain of the Fc domain as set forth in SEQ ID No: 5 while the IL-15Rα protein domain is operably linked to the other chain of the Fc domain as set forth in SEQ ID No: 6; in some other embodiments, the IL-15 variant is operably linked to one chain of the Fc domain as set forth in SEQ ID No: 6 while the IL-15Rα protein domain is operably linked to the other chain of the Fc domain as set forth in SEQ ID No: 5; in certain embodiments, the IL-15 variant is operably linked to one chain of the Fc domain as set forth in SEQ ID No: 7 while the IL-15Rα protein domain is operably linked to the other chain of the Fc domain as set forth in SEQ ID No: 8; in some other embodiments, the IL-15 variant is operably linked to one chain of the Fc domain as set forth in SEQ ID No: 8 while the IL-15Rα protein domain is operably linked to the other chain of the Fc domain as set forth in SEQ ID No: 7.

In some exemplary embodiments, the IL-15/IL-15Rα-Fc polypeptide complexes as disclosed herein comprise two chains, the first chain comprises an IL-15 variant comprising a truncation of 4 amino acids from the C terminal operably linked to an IgG4 Fc domain (V1 format), the second chain comprises a IL-15Rα protein operably linked to the other chain of the IgG4 Fc domain (V1 format). Alternatively, the Fc domain may be in V322, V805 or V803 format.

In some exemplary embodiments, the IL-15/IL-15Rα-Fc polypeptide complexes as disclosed herein comprise two chains, the first chain comprises an IL-15 variant comprising a truncation of 5 amino acids from the C terminal operably linked to an IgG4 Fc domain (V1 format), the second chain comprises a IL-15Rα protein operably linked to the other chain of the IgG4 Fc domain (V1 format). Alternatively, the Fc domain may be in V322, V805 or V803 format.

In some exemplary embodiments, the IL-15/IL-15Rα-Fc polypeptide complexes as disclosed herein comprise two chains, the first chain comprises an IL-15 variant comprising a truncation of 6 amino acids from the C terminal operably linked to an IgG4 Fc domain (V1 format), the second chain comprises a IL-15Rα protein operably linked to the other chain of the IgG4 Fc domain (V1 format). Alternatively, the Fc domain may be in V322, V805 or V803 format.

In some exemplary embodiments, the IL-15/IL-15Rα-Fc polypeptide complexes as disclosed herein comprise two chains, the first chain comprises an IL-15 variant comprising a truncation of 7 amino acids from the C terminal operably linked to an IgG4 Fc domain (V1 format), the second chain comprises a IL-15Rα protein operably linked to the other chain of the IgG4 Fc domain (V1 format). Alternatively, the Fc domain may be in V322, V805 or V803 format.

In some exemplary embodiments, the IL-15/IL-15Rα-Fc polypeptide complexes as disclosed herein comprise two chains, the first chain comprises an amino acid sequence as set forth in SEQ ID No: 13, 15, 17, 19, 21, 23, 25, 27, 29, 31, 33, 35, 37, 39, 41, 43, 45, 47, 49, 70, 72, or 74, and/or the second chain comprises an amino acid sequence as set forth in SEQ ID No: 14, 16, 18, 20, 24, 26, 28, 30, 32, 34, 36, 38, 40, 42, 46, 48, 50, 71, 73, or 75. In some exemplary embodiments, the IL-15/IL-15Rα-Fc polypeptide complexes as disclosed herein comprise two chains selected from the following pairs: amino acid sequences as set forth in SEQ ID No: 13/14, 15/16, 17/18, 19/20, 21/22, 23/24, 25/26, 27/28, 29/30, 31/32, 33/34, 35/36, 37/38, 39/40, 41/42, 43/44, 45/46, 47/48, 49/50, 70/71, 72/73, or 74/75.

In some exemplary embodiments, the IL-15/IL-15Rα-Fc polypeptide complexes as disclosed herein comprise two chains, the first chain comprises an amino acid sequence as set forth in SEQ ID No: 19, 21, 71, 73, or 75, and/or the second chain comprises an amino acid sequence as set forth in SEQ ID No: 20, 71, 73, or 75. Specifically, the IL-15/IL-15Rα-Fc polypeptide complexes comprise a pair of two chains selected from 19/20, 70/71, 72/73, or 74/75.

### Fc Domain

As described above, single chain IL15-Fc fusion protein and IL15Rα-Fc protein each comprises one chain of a Fc domain, which associates together to form the Fc dimeric domain, similar to the Fc region in an antibody.

The Fc domain may be a wild-type Fc or an Fc variant. A wild-type Fc may be a human IgG1, IgG2, IgG3 or IgG4 Fc. In some embodiments, the wild-type Fc is a human IgG1 Fc or IgG4 Fc. The Fc variant comprises one or more amino acid residue modifications (e.g. substitutions, insertions and/or deletions) compared to the wild-type Fc (e.g., human IgG1, IgG2, IgG3 or IgG4 Fc). In some embodiments, the Fc variant comprises one or more amino acid residue modifications (e.g. substitutions, insertions and/or deletions) compared to wild-type human IgG1 Fc or IgG4 Fc.

Said one or more amino acid modifications comprised in the Fc variant may alter the binding to one or more FcγR receptors, alter the binding to FcRn receptors, etc. In certain embodiments, the Fc domain of the heterodimeric polypeptide complexes comprises one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody and antigen-binding fragment thereof to improve binding affinity with FcRn are well-known in the art, see, for example, Vaughn, D. et al, Structure, 6(1): 63-73, 1998; Kontermann, R. et al, Antibody Engineering, Volume 1, Chapter 27: Engineering of the Fc region for improved PK, published by Springer, 2010; Yeung, Y. et al, Cancer Research, 70: 3269-3277 (2010); and Hinton, P. et al, J. Immunology, 176:346-356 (2006).

The two chains of the Fc domain may associate together via a disulfide bond. In some embodiments, the Fc domain comprises one or more amino acid modifications (e.g. substitutions) in the interface of the Fc region to facilitate and/or promote heterodimerization. For example, the two chains of the Fc domain are engineered to comprise a “knob-into-hole” structure to promote heterodimerization, which includes introduction of a protuberance (“knob”) into a first Fc polypeptide and a cavity (“hole”) into a second Fc polypeptide, wherein the protuberance can be positioned in the cavity so as to promote interaction of the first and second Fc polypeptides to form a heterodimer or a complex. Methods of generating antibodies with these modifications are known in the art, e.g., as described in U.S. Pat. No. 5,731,168. Specifically, the Fc domain may comprise at least one “knob” (protuberance) and at least one “hole” (cavity), wherein presence of the “knob” and “hole” enhances formation of a complex or heterodimer (for more detail see WO 2005/063816). In some embodiments, the Fc domain as disclosed herein comprises a first and a second Fc polypeptide chain, wherein the first and second polypeptide each comprises one or more mutations with respect to wild type human IgG1 Fc. The IL-15 domain may be fused to one chain of the Fc domain comprising a “knob” mutation while the IL-15Rα domain is fused to the other chain comprising a “hole” mutation, or vice versa. In at least one embodiment, a “hole” mutation is Y349C, T366S, L368A, and/or Y407V, and a “knob” mutation is S354C and/or T366W.

In certain embodiments, the Fc domain of the fusion proteins comprise one or more amino acid substitution(s) that alters the antibody-dependent cellular cytotoxicity (ADCC) and/or complement dependent cytotoxicity (CDC). Certain amino acid residues at CH2 domain of the Fc region can be substituted to provide for reduced ADCC activity.

In certain embodiments, the Fc domain is an IgG4 Fc and comprises a S228P mutation. As known in the art, S228P mutation in an antibody may serve to remove Fab arm exchange and improve stability. In certain embodiments, the Fc domain comprises a triple mutation M252Y/S254T/T256E (“YTE”). This triple mutation has been reported to cause an about 10-fold increase in binding to human neonatal Fc receptor (FcRn) and an almost 4-fold increase in the serum half-life of YTE-containing human IgGs in cynomolgus monkeys (Oganesyan V. et al, Mol Immunol. 2009 May; 46(8-9):1750-5). In some further embodiments, the Fc domain comprises additional mutations to alter the interaction between Fc and human FcRn. In some specific embodiments, the Fc domain comprises C220S, G236R, and/or L328R substitution(s) in addition to the “YTE” triple mutation. A double “G236R/L328R” or “RR” substitution has been reported to reduce binding affinity to FcγR and the Fc effector function (ADCC, CDC, ADCP). In some embodiments, the Fc domain comprises a FALA mutation (F234A/L235A) to abrogate the binding with Fc receptors or complement receptors.

The “EU numbering system” or “EU index” is generally used when referring to a residue in an immunoglobulin heavy chain constant region (e.g., the EU index reported in Kabat et al. Sequences of Proteins of Immunological Interest (5^{th} Ed.), US Dept. of Health and Human Services, PHS, NIH, NIH Publication no. 91-3242). The “EU numbering as in Kabat” or “EU index as in Kabat” refers to the residue numbering of the human IgG1 EU antibody. Unless stated otherwise herein, references to residue numbers in the constant domain of Fc regions means residue numbering by the EU numbering system.

In some specific embodiments, the Fc variant comprises two chains, wherein the amino acid sequence of the first chain has at least 80%, e.g. 80%, 85%, 90%, 95% or more (e.g. 100%) sequence identity to SEQ ID No: 3, and/or the amino acid sequence of the second chain has at least 80%, e.g. 80%, 85%, 90%, 95% or more (e.g. 100%) sequence identity to SEQ ID No: 4. In some specific embodiments, the first chain of the Fc variant comprises or consists of an amino acid sequence as set forth in SEQ ID No: 3, and the second chain of the Fc variant comprises or consists of an amino acid sequence as set forth in SEQ ID No: 4.

In some specific embodiments, the Fc variant comprises two chains, wherein the amino acid sequence of the first chain has at least 80%, e.g. 80%, 85%, 90%, 95% or more (e.g. 100%) sequence identity to SEQ ID No: 5, and/or the amino acid sequence of the second chain has at least 80%, e.g. 80%, 85%, 90%, 95% or more (e.g. 100%) sequence identity to SEQ ID No: 6. In some specific embodiments, the first chain of the Fc variant comprises or consists of an amino acid sequence as set forth in SEQ ID No: 5, and the second chain of the Fc variant comprises or consists of an amino acid sequence as set forth in SEQ ID No: 6.

In some specific embodiments, the Fc variant comprises two chains, wherein the amino acid sequence of the first chain has at least 80%, e.g. 80%, 85%, 90%, 95% or more (e.g. 100%) sequence identity to SEQ ID No: 7, and/or the amino acid sequence of the second chain has at least 80%, e.g. 80%, 85%, 90%, 95% or more (e.g. 100%) sequence identity to SEQ ID No: 8. In some specific embodiments, the first chain of the Fc variant comprises or consists of an amino acid sequence as set forth in SEQ ID No: 7, and the second chain of the Fc variant comprises or consists of an amino acid sequence as set forth in SEQ ID No: 8.

The expression of “at least 80%” with regard to sequence identity may include e.g. 80%, 85%, 88%, 90%, 91%, 92%, 93%, 94%, 95%, 96%, 97%, 98%, 99%, 100% (i.e. the same). The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percentage of identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally, or alternatively, the protein sequences of the present disclosure can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the fusion protein molecules of the disclosure. To obtain gapped alignments for comparison purposes, Gapped BLAST can be utilized as described in Altschul et al, (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

### Properties of the IL-15 Variants and IL-15-Fc Fusion Proteins as Disclosed Herein

The present disclosure provides potency reduced, PK/PD enhanced IL-15 variants and IL-15-Fc polypeptide complexes comprising these IL-15 variants. The IL-15 variants have shown good thermal stability, reduced potency/toxicity, prolonged PK, enhanced PD, thus may serve as a novel immunotherapy agent with improved anti-tumor efficacy.

The functionality of these IL-15 variants and IL-15-Fc fusion proteins may be assessed in a number of ways, such as in vitro or in vivo assays.

In some embodiments, the effect of the IL-15 variants and IL-15-Fc polypeptide complexes is evaluated by immune cell proliferation assays, using for example Ki-67 intracellular staining of immune effector cells. Ki-67 is a protein strictly associated with cell proliferation and the percentage of Ki-67 on CD8+T and NK cells may be measured by FACS, which indicates their activities in stimulating CD8+T and NK cells. Other immune cells can also be used for evaluating the effect on cell proliferation, such as CD45+ lymphocytes, among others.

In some embodiments, the effect of the IL-15 variants and IL-15-Fc polypeptide complexes may be evaluated by quantifying a signaling pathway measured by phosphorylation of certain factors, such as STAT5 phosphorylation. In some embodiments, the effect of the IL-15 variants and IL-15-Fc polypeptide complexes may be evaluated by assessing T cell activity measured by cytokine production. In some embodiments, the effect of the IL-15 variants and IL-15-Fc fusion proteins may be evaluated by assessing lympho-expansion (counts in white blood cells, lymphocytes, NK cells, T cells and T cell subsets, monocytes, and basophile).

The IL-15 comprising polypeptide complexes and fusion proteins of the present disclosure provide at least one of the following properties:
(a) more moderate potency in simulating immune cell proliferation, such as CD8+ T cell and NK cell proliferation in human, cyno and mouse;
(b) good thermo stability;
(c) prolonged pharmacokinetics profile compared to wild-type IL-15 or IL-15 comprising polypeptide complexes and fusion proteins;
(d) shows a prolonged and higher lymphocyte proliferation in pharmacodynamics study;
(e) no obvious toxicity in vivo.

### Nucleic Acid Molecules Encoding the IL-15 Variants and Fusion Proteins

In some aspects, the disclosure is directed to an isolated nucleic acid molecule, comprising a nucleic acid sequence encoding the IL-15 variant or the heterodimeric Fc fusion protein as disclosed herein.

Nucleic acids of the disclosure can be obtained using standard molecular biology techniques. The isolated nucleic acid encoding the IL-15 variant can be operatively linked to another DNA molecule encoding an Fc domain. Similarly, a nucleic acid encoding the IL-15Rα domain (wild-type, variant or any fragment thereof that retains IL15 binding activity) can be operatively linked to another DNA molecule encoding an Fc domain. DNA fragments encompassing these regions can be obtained by standard PCR amplification.

Once DNA fragments encoding IL-15, IL-15Rα and Fc domains are obtained, these DNA fragments can be further manipulated by standard recombinant DNA techniques, for example incorporated into expression vectors as is known in the art. In some embodiments, nucleic acids encoding these DNA fragments are each contained within a single expression vector, generally under different or the same promoter control. In some other embodiments, nucleic acids encoding these DNA fragments are operably linked and contained in a single expression vector under the control of the same promoter. The term “operatively linked”, as used in this context, is intended to mean that the two DNA fragments are joined such that the amino acid sequences encoded by the two DNA fragments remain in-frame.

### Vectors and Host Cells

The DNAs encoding the IL-15 variant or IL-15 comprising fusion proteins may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of desired proteins in the recombinant host cells. See, e.g., PCT Publication No. WO 87/04462.

Generally, a nucleic acid sequence encoding one or all chains of the fusion protein can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter. Regulatable promoters that include a repressor with the operon can be used.

Host cells as disclosed in the present disclosure may be any cell which is suitable for expressing the fusion proteins of the present disclosure, for instance, bacterial cells, yeast, mammalian cells. Mammalian host cells for expressing the fusion proteins of the present disclosure include Chinese Hamster Ovary (CHO cells) (including dhfr CHO cells, described in Urlaub and Chasin, (1980) Proc. Natl. Acad. ScL USA 77:4216-4220, used with a DHFR selectable marker, e.g., as described in R. J. Kaufman and P. A. Sharp (1982) J. Mol. Biol. 159:601-621), NSO myeloma cells, COS cells and SP2 cells.

In particular, for use with NSO myeloma cells, another expression system is the GS gene expression system disclosed in WO 87/04462, WO 89/01036 and EP 338,841. When recombinant expression vectors encoding the antibody are introduced into mammalian host cells, the fusion proteins are produced by culturing the host cells for a period of time sufficient to allow for expression of the fusion protein in the host cells or, secretion of the fusion protein into the culture medium in which the host cells are grown. The fusion proteins can be recovered from the culture medium using standard protein purification methods.

### Pharmaceutical Compositions

In some aspects, the disclosure is directed to a pharmaceutical composition comprising the heterodimeric polypeptide complex as disclosed herein and a pharmaceutically acceptable carrier. In some aspects, the disclosure is directed to a pharmaceutical composition comprising the fusion proteins as disclosed herein and a pharmaceutically acceptable carrier. In some aspects, the disclosure is directed to a pharmaceutical composition comprising a nucleic acid molecule encoding the heterodimeric polypeptide complex or the fusion protein as disclosed herein and a pharmaceutically acceptable carrier.

### Components of the Compositions

The pharmaceutical composition may optionally contain one or more additional pharmaceutically active ingredients, such as an antibody. The pharmaceutical compositions of the disclosure also can be administered in a combination therapy with, for example, another immune-stimulatory agent, anti-cancer agent, an antiviral agent, or a vaccine. A pharmaceutically acceptable carrier can include, for example, a pharmaceutically acceptable liquid, gel or solid carriers, an aqueous medium, a non-aqueous medium, an anti-microbial agent, isotonic agents, buffers, antioxidants, anesthetics, suspending/dispersing agent, a chelating agent, a diluent, adjuvant, excipient or a nontoxic auxiliary substance, other known in the art various combinations of components or more.

Suitable components may include, for example, antioxidants, fillers, binders, disintegrating agents, buffers, preservatives, lubricants, flavorings, thickening agents, coloring agents, emulsifiers or stabilizers such as sugars and cyclodextrin. Suitable anti-oxidants may include, for example, methionine, ascorbic acid, EDTA, sodium thiosulfate, platinum, catalase, citric acid, cysteine, mercapto glycerol, thioglycolic acid, Mercapto sorbitol, butyl methyl anisole, butylated hydroxy toluene and/or propylgalacte. As disclosed in the present disclosure, the composition may include one or more anti-oxidants such as methionine, reducing antibody or antigen binding fragment thereof that may be oxidized. The oxidation reduction may prevent or reduce a decrease in binding affinity, thereby enhancing protein stability and extended shelf life. Thus, in some embodiments, the present disclosure provides a composition comprising fusion proteins and one or more anti-oxidants such as methionine. The present disclosure further provides a variety of methods, wherein a fusion protein is mixed with one or more anti-oxidants, such as methionine, so that the fusion protein can be prevented from oxidation, to extend their shelf life and/or increased activity.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

### Administration, Formulation and Dosage

The pharmaceutical composition of the disclosure may be administered in vivo, to a subject in need thereof, by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilizers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Similarly, the particular dosage regimen, including dose, timing and repetition, will depend on the particular individual and that individual's medical history, as well as empirical considerations such as pharmacokinetics (e.g., half-life, clearance rate, etc.).

Frequency of administration may be determined and adjusted over the course of therapy, and is based on reducing the number of proliferative or tumorigenic cells, maintaining the reduction of such neoplastic cells, reducing the proliferation of neoplastic cells, or delaying the development of metastasis. In some embodiments, the dosage administered may be adjusted or attenuated to manage potential side effects and/or toxicity. Alternatively, sustained continuous release formulations of a subject therapeutic composition may be appropriate.

It will be appreciated by one of skill in the art that appropriate dosages can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieve the desired effect without causing substantial harmful or deleterious side-effects.

In general, the IL-15/IL-15Rα-Fc fusion proteins of the disclosure may be administered in various ranges. These include about 100 μg/kg body weight to about 10 mg/kg body weight per dose; about 100 μg/kg body weight to about 1 mg/kg body weight per dose; about 1 g/kg body weight to about 10 mg/kg body weight per dose. Other ranges include about 100 μg/kg body weight to about 200 μg/kg body weight per dose; about 200 μg/kg body weight to about 300 μg/kg body weight per dose; about 300 μg/kg body weight to about 400 μg/kg body weight per dose; about 400 μg/kg body weight to about 0.5 μg/kg body weight per dose; and about 0.5 mg/kg body weight to about 1 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight.

In any event, the IL-15/IL-15Rα-Fc fusion proteins of the disclosure is preferably administered as needed to subjects in need thereof. Determination of the frequency of administration may be made by persons skilled in the art, such as an attending physician based on considerations of the condition being treated, age of the subject being treated, severity of the condition being treated, general state of health of the subject being treated and the like.

In certain preferred embodiments, the course of treatment involving the IL-15/IL-15Rα-Fc fusion proteins of the present disclosure will comprise multiple doses of the selected drug product over a period of weeks or months. More specifically, the IL-15/IL-15Rα-Fc fusion proteins of the present disclosure may be administered once every four days, every week, every ten days, every two weeks, every three weeks, every month, every six weeks, every two months, every ten weeks or every three months. In this regard, it will be appreciated that the dosages may be altered or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In selected embodiments, the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

Compatible formulations for parenteral administration (e.g., intravenous injection) will comprise the IL-15/IL-15Rα-Fc fusion proteins as disclosed herein in concentrations that are considered suitable for administration and can be empirically determined by those skilled in the art, for example from about 10 μg/ml to about 10 mg/ml.

### Applications of the Disclosure

The IL-15/IL-15Rα-Fc fusion proteins, pharmaceutical compositions and methods of the present disclosure have numerous in vitro and in vivo utilities involving, for example, enhancement of immune response. For example, these molecules can be administered to cells in culture, in vitro or ex vivo, or to human subjects, e.g., in vivo, to enhance immunity in a variety of situations. The immune response can be modulated, for instance, augmented, stimulated or up-regulated.

For instance, the subjects include human patients in need of enhancement of an immune response. The methods are particularly suitable for treating human patients having a disorder that can be treated by augmenting an immune response (e.g., the NK/T-cell mediated immune response). In a particular embodiment, the methods are particularly suitable for treatment of cancer in vivo. To achieve enhancement of immunity, the IL-15/IL-15Rα-Fc fusion proteins can be administered alone or in combination with another therapy. When IL-15/IL-15Rα-Fc fusion proteins are administered together with another agent, the two can be administered in either order or simultaneously.

### Treatment of Disorders Including Cancers and Infectious Diseases

In some aspects, the present disclosure provides a method of treating a disorder or a disease in a mammal, which comprises administering to the subject (for example, a human) in need of treatment a therapeutically effective amount of the IL-15/IL-15Rα-Fc fusion proteins as disclosed herein. The disorder or disease may be a cancer.

A variety of cancers, whether malignant or benign and whether primary or secondary, may be treated or prevented with a method provided by the disclosure. The cancers may be solid cancers or hematologic malignancies. Examples of such cancers include lung cancers such as bronchogenic carcinoma (e.g., non-small cell lung cancer, squamous cell carcinoma, small cell carcinoma, large cell carcinoma, and adenocarcinoma), alveolar cell carcinoma, bronchial adenoma, chondromatous hamartoma (noncancerous), and sarcoma (cancerous); heart cancer such as myxoma, fibromas, and rhabdomyomas; bone cancers such as osteochondromas, condromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain cancer such as gliomas (e.g., glioblastoma multiforme), anaplastic astrocytomas, astrocytomas, oligodendrogliomas, medulloblastomas, chordoma, Schwannomas, ependymomas, meningiomas, pituitary adenoma, pinealoma, osteomas, hemangioblastomas, craniopharyngiomas, chordomas, germinomas, teratomas, dermoid cysts, and angiomas; cancers in digestive system such as colon cancer, leiomyoma, epidermoid carcinoma, adenocarcinoma, leiomyosarcoma, stomach adenocarcinomas, intestinal lipomas, intestinal neurofibromas, intestinal fibromas, polyps in large intestine, and colorectal cancers; liver cancers such as hepatocellular adenomas, hemangioma, hepatocellular carcinoma, fibrolamellar carcinoma, cholangiocarcinoma, hepatoblastoma, and angiosarcoma; kidney cancers such as kidney adenocarcinoma, renal cell carcinoma, hypernephroma, and transitional cell carcinoma of the renal pelvis; bladder cancers; hematological cancers such as acute lymphocytic (lymphoblastic) leukemia, acute myeloid (myelocytic, myelogenous, myeloblasts, myelomonocytic) leukemia, chronic lymphocytic leukemia (e.g., Sezary syndrome and hairy cell leukemia), chronic myelocytic (myeloid, myelogenous, granulocytic) leukemia, Hodgkin's lymphoma, non-Hodgkin's lymphoma, B cell lymphoma, mycosis fungoides, and myeloproliferative disorders (including myeloproliferative disorders such as polycythemia vera, myelofibrosis, thrombocythemia, and chronic myelocytic leukemia); skin cancers such as basal cell carcinoma, squamous cell carcinoma, melanoma, Kaposi's sarcoma, and Paget's disease; head and neck cancers; eye-related cancers such as retinoblastoma and intraoccular melanocarcinoma; male reproductive system cancers such as benign prostatic hyperplasia, prostate cancer, and testicular cancers (e.g., seminoma, teratoma, embryonal carcinoma, and choriocarcinoma); breast cancer; female reproductive system cancers such as uterine cancer (endometrial carcinoma), cervical cancer (cervical carcinoma), cancer of the ovaries (ovarian carcinoma), vulvar carcinoma, vaginal carcinoma, fallopian tube cancer, and hydatidiform mole; thyroid cancer (including papillary, follicular, anaplastic, or medullary cancer); pheochromocytomas (adrenal gland); noncancerous growths of the parathyroid glands; pancreatic cancers; and hematological cancers such as leukemias, myelomas, non-Hodgkin's lymphomas, and Hodgkin's lymphomas. In a specific embodiment, the cancer is colon cancer.

In some embodiments, examples of cancer include but not limited to B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), B-cell proliferative disorders, and Meigs' syndrome. More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B-cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B-cell lymphoma, splenic marginal zone lymphoma, extranodal marginal zone-MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and/or Sezary syndrome, skin (cutaneous) lymphomas, anaplastic large cell lymphoma, angiocentric lymphoma.

In some embodiments, examples of cancer further include, but are not limited to, B-cell proliferative disorders, which further include, but are not limited to, lymphomas (e.g., B-Cell Non-Hodgkin's lymphomas (NHL)) and lymphocytic leukemias. Such lymphomas and lymphocytic leukemias include e.g. a) follicular lymphomas, b) Small Non-Cleaved Cell Lymphomas/Burkitt's lymphoma (including endemic Burkitt's lymphoma, sporadic Burkitt's lymphoma and Non-Burkitt's lymphoma), c) marginal zone lymphomas (including extranodal marginal zone B-cell lymphoma (Mucosa-associated lymphatic tissue lymphomas, MALT), nodal marginal zone B-cell lymphoma and splenic marginal zone lymphoma), d) Mantle cell lymphoma (MCL), e) Large Cell Lymphoma (including B-cell diffuse large cell lymphoma (DLCL), Diffuse Mixed Cell Lymphoma, Immunoblastic Lymphoma, Primary Mediastinal B-Cell Lymphoma, Angiocentric Lymphoma-Pulmonary B-Cell Lymphoma), f) hairy cell leukemia, g) lymphocytic lymphoma, Waldenstrom's macroglobulinemia, h) acute lymphocytic leukemia (ALL), chronic lymphocytic leukemia (CLL)/small lymphocytic lymphoma (SLL), B cell prolymphocytic leukemia, i) plasma cell neoplasms, plasma cell myeloma, multiple myeloma, plasmacytoma, and/or j) Hodgkin's disease.

In some embodiments, the IL-15 comprising fusion proteins as disclosed herein may be used to treat or prevent an infectious disease, which may be caused by a viral, bacterial, fungal or parasite infection. In some other embodiments, the IL-15 comprising fusion proteins as disclosed herein may be used to treat or prevent immunodeficiency or lymphopenia.

Stimulation of an Immune Response without Incurring Cytotoxicity

In some aspects, the disclosure also provides a method of enhancing (for example, stimulating) an immune response in a subject comprising administering an IL-15/IL-15Rα-Fc fusion protein of the disclosure to the subject such that an immune response in the subject is enhanced while no undesired side effects are presented. For example, the subject is a mammal. In a specific embodiment, the subject is a human.

The term “enhancing an immune response” or its grammatical variations, means stimulating, evoking, increasing, improving, or augmenting any response of a mammal's immune system. The immune response may be a cellular response (i.e. cell-mediated, such as cytotoxic T lymphocyte mediated) or a humoral response (i.e. antibody mediated response), and may be a primary or secondary immune response. Examples of enhancement of immune response include increased CD4⁺ helper T cell activity and generation of cytolytic T cells. The enhancement of immune response can be assessed using a number of in vitro or in vivo measurements known to those skilled in the art, including, but not limited to, cytotoxic T lymphocyte assays, release of cytokines (for example IL-2 production or IFN-γ production), regression of tumors, survival of tumor bearing animals, antibody production, immune cell proliferation, expression of cell surface markers, and cytotoxicity. Typically, methods of the disclosure enhance the immune response by a mammal when compared to the immune response by an untreated mammal or a mammal not treated using the methods as disclosed herein. In one embodiment, the immune response is cytokine production, particularly IFN-γ production or IL-12 production. In another embodiment, the immune response is enhanced B cell proliferation.

The IL-15 fusion proteins as disclosed herein may be used alone as a monotherapy, or more often, used in combination with cell immunotherapies, targeted therapies, chemical therapies or radiotherapies.

### Application in Gene Therapy

In some embodiments, IL-15 variants and IL-15 variant fusion proteins or nucleic acid molecules encoding them as disclosed herein are used in gene therapy. The gene coding IL-15 variants and IL-15 variant fusion proteins are integrated into therapeutic vectors, such as lentivirus, AAV, poxvirus, herpes zoster virus, oncolytic virus and other RNA/DNA vectors.

### Application in Cellular Immunotherapies

In some embodiments, the IL-15 variants and IL-15 variant fusion proteins as disclosed herein are used in combination with a cellular immunotherapy, also known as adoptive cell therapy. As is generally known, cellular immunotherapy is a form of treatment that uses the cells of human body's immune system to eliminate cancer. Some of these approaches involve directly isolating our own immune cells and simply expanding their numbers (e.g. performed by activating and expanding the immune cells of patient outside of the body and infused into the patient), whereas others involve genetically engineering immune cells (via gene therapy) to enhance their cancer-fighting capabilities. Cellular immunotherapies can be deployed in different ways, including but not limited to Tumor-Infiltrating Lymphocyte (TIL) therapy, Engineered T Cell Receptor (TCR-T) therapy, Chimeric Antigen Receptor (CAR) T Cell therapy, chimeric Antigen Receptor (CAR) γδT Cell therapy, CAR NK Cell therapy, CAR Macrophage Cell, Natural Killer (NK) Cell therapy.

In some embodiments, IL-15 variants and IL-15 variant fusion proteins as disclosed herein are integrated in cellular therapy, whereas the gene coding IL-15 variants and IL-15 variant fusion proteins are integrated into the genome of the cells by genetically engineering immune cells.

Combined Use with Gene Therapies

In some embodiments, the IL-15 variants and IL-15 variant fusion proteins as disclosed herein are used in combination with a gene therapy. The gene coding IL-15 variants and IL-15 variant fusion proteins may be delivered to a subject by therapeutic vectors, such as viruses, including lentivirus, AAV, poxvirus, herpes zoster virus, oncolytic virus. Transfer of gene may be performed through transformation where under specific conditions the gene is directly taken up by the bacterial cells, transduction where a bacteriophage is used to transfer the genetic material and lastly transfection that involves forceful delivery of gene using either viral or non-viral vectors. The non-viral transfection methods are subdivided into physical, chemical and biological. The physical methods include electroporation, biolistic, microinjection, laser, elevated temperature, ultrasound and hydrodynamic gene transfer. The chemical methods utilize calcium-phosphate, DAE-dextran, liposomes and nanoparticles for transfection. The biological methods are increasingly using viruses for gene transfer, these viruses could either integrate within the genome of the host cell conferring a stable gene expression, whereas few other non-integrating viruses are episomal and their expression is diluted proportional to the cell division.

Combined Use with Cellular Immunotherapies

In some embodiments, the IL-15 variants and IL-15 variant fusion proteins as disclosed herein are used in combination with a cellular immunotherapy, also known as adoptive cell therapy. As is generally known, cellular immunotherapy is a form of treatment that uses the cells of human body's immune system to eliminate cancer. Some of these approaches involve directly isolating our own immune cells and simply expanding their numbers (e.g. performed by activating and expanding the immune cells of patient outside of the body and infused into the patient), whereas others involve genetically engineering immune cells (via gene therapy) to enhance their cancer-fighting capabilities. Cellular immunotherapies can be deployed in different ways, including but not limited to Tumor-Infiltrating Lymphocyte (TIL) therapy, Engineered T Cell Receptor (TCR-T) therapy, Chimeric Antigen Receptor (CAR) T Cell therapy, CAR NK Cell therapy, CAR Macrophage Cell therapy, Natural Killer (NK) Cell therapy.

Combined Use with Targeted Therapies and Chemotherapies

The heterodimeric polypeptide complexes as disclosed herein may be administered as the sole active ingredient or in conjunction with, e.g. as an adjuvant to or in combination to, other drugs e.g. anti-cancer agents, immunomodulating agents or other anti-inflammatory agents, e.g. for the treatment or prevention of diseases mentioned above.

For the purposes of the present disclosure a “chemotherapeutic agent” comprises a chemical compound that non-specifically decreases or inhibits the growth, proliferation, and/or survival of cancer cells (e.g., cytotoxic or cytostatic agents). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapeutic agents can include any chemical agent that inhibits, or is designed to inhibit, a cancerous cell or a cell likely to become cancerous or generate tumorigenic progeny (e.g., TIC). Such agents are often administered, and are often most effective, in combination, e.g., in regimens such as CHOP or FOLFIRI. Examples of anti-cancer agents that may be used in combination with the proteins of the present disclosure (either as a component of conjugate or in an unconjugated state) include, but are not limited to, e.g. paclitaxel, gemcitabine, cisplatinum, doxorubicin, 5-fluorouracil, capecitabine, combretastatin, leucovorin etc.

For example, the heterodimeric polypeptide complexes as described herein may be used in combination with DMARD, e.g. Gold salts, sulphasalazine, antimalarias, methotrexate, D-penicillamine, azathioprine, mycophenolic acid, cyclosporine A, tacrolimus, sirolimus, minocycline, lefiunomide, glococorticoids; a calcineurin inhibitor, e.g. cyclosporin A or FK 506; a modulator of lymphocyte recirculation, e.g. FTY720 and FTY720 analogs; a mTOR inhibitor, e.g. rapamycin, 40-O-(2-hydroxyethyl)-rapamycin, CCI779, ABT578, AP23573 or TAFA-93; an ascomycin having immunosuppressive properties, e.g. ABT-281, ASM981, etc.; corticosteroids; cyclo-phosphamide; azathioprene; methotrexate; lefiunomide; mizoribine; mycophenolic acid; myco-phenolate mofetil; 15-deoxyspergualine or an immunosuppressive homologue, analogue or derivative thereof; immunosuppressive

It will be appreciated that, in selected embodiments as discussed above, such anti-cancer agents may comprise conjugates and may be associated with the disclosed IL-15/IL-15Rα-Fc fusion proteins prior to administration. More specifically, in certain embodiments selected anti-cancer agents will be linked to the unpaired cysteines of the engineered IL-15/IL-15Rα-Fc fusion proteins to provide engineered conjugates as set forth herein. Accordingly, such engineered conjugates are expressly contemplated as being within the scope of the present disclosure. In other embodiments, the disclosed anti-cancer agents will be given in combination with site-specific conjugates comprising a different therapeutic agent as set forth above.

It will be easily appreciated that, the anti-cancer agents or immunomodulating agents to be used in combination with the IL-15 fusion proteins as disclosed herein should be compatible with the IL-15 fusion proteins, i.e. would not reduce, disturb, or eliminate the effect of the IL-15 fusion proteins as disclosed herein, and preferably provide a coordinating or even synergistic effect
Combined Use with Radiotherapies

The present disclosure also provides for the combination of the IL-15/IL-15Rα-Fc fusion proteins thereof with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed fusion proteins and polypeptide complexes may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

### Pharmaceutical Packs and Kits

Pharmaceutical packs and kits comprising one or more containers, comprising one or more doses of the IL-15/IL-15Rα fusion proteins are also provided. In certain embodiments, a unit dosage is provided wherein the unit dosage contains a predetermined amount of a composition comprising the IL-15/IL-15Rα fusion proteins, with or without one or more additional agents. For other embodiments, such a unit dosage is supplied in single-use prefilled syringe for injection. The composition contained in the unit dosage may comprise saline, sucrose, or the like; a buffer, such as phosphate, or the like; and/or be formulated within a stable and effective pH range. Alternatively, in certain embodiments, the composition may be provided as a lyophilized powder that may be reconstituted upon addition of an appropriate liquid, for example, sterile water or saline solution. In certain preferred embodiments, the composition comprises one or more substances that inhibit protein aggregation, including, but not limited to, sucrose and arginine. Any label on, or associated with, the container(s) indicates that the enclosed composition is used for treating the disease condition of choice.

The present disclosure also provides kits for producing single-dose or multi-dose administration units of proteins and, optionally, one or more anti-cancer agents. The kit comprises a container and a label or package insert on or associated with the container. Suitable containers include, for example, bottles, vials, syringes, etc. The containers may be formed from a variety of materials such as glass or plastic and contain a pharmaceutically effective amount of the disclosed proteins. The container(s) may comprise a sterile access port (for example the container may be an intravenous solution bag or a vial having a stopper pierceable by a hypodermic injection needle). Such kits will generally contain in a suitable container a pharmaceutically acceptable formulation of the engineered proteins and, optionally, one or more anti-cancer agents in the same or different containers. The kits may also contain other pharmaceutically acceptable formulations, either for diagnosis or combined therapy. For example, in addition to the IL-15/IL-15Rα fusion proteins of the disclosure such kits may contain any one or more of a range of anti-cancer agents such as chemotherapeutic or radiotherapeutic drugs; anti-angiogenic agents; anti-metastatic agents; targeted anti-cancer agents; cytotoxic agents; and/or other anti-cancer agents.

More specifically the kits may have a single container that contains the disclosed the IL-15/IL-15Rα-Fc fusion proteins, with or without additional components, or they may have distinct containers for each desired agent. Where combined therapeutics are provided for conjugation, a single solution may be pre-mixed, either in a molar equivalent combination, or with one component in excess of the other. Alternatively, the IL-15/IL-15Rα-Fc fusion proteins and any optional anti-cancer agent of the kit may be maintained separately within distinct containers prior to administration to a patient. The kits may also comprise a second/third container means for containing a sterile, pharmaceutically acceptable buffer or other diluents such as bacteriostatic water for injection (BWFI), phosphate-buffered saline (PBS), Ringer's solution and dextrose solution.

When the components of the kit are provided in one or more liquid solutions, the liquid solution is preferably an aqueous solution, with a sterile aqueous or saline solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer the IL-15/IL-15Rα fusion proteins and any optional components to a patient, e.g., one or more needles, I.V. bags or syringes, or even an eye dropper, pipette, or other such like apparatus, from which the formulation may be injected or introduced into the animal or applied to a diseased area of the body. The kits of the present disclosure will also typically include a means for containing the vials, or such like, and other component in close confinement for commercial sale, such as, e.g., injection or blow-molded plastic containers into which the desired vials and other apparatus are placed and retained.

### Sequence Listing Summary

The following Table A provides the sequences of the proteins, variants and exemplary polypeptide complexes disclosed herein. “W327151-TXU1.P1-V0013.uIgG4V1” and “W327151-TXU1.P1-V0013.uIgG4V322” can be abbreviated as “V0013.uIgG4V1” and “0013.uIgG4V322”, respectively, and similarly applied to others.

TABLE A

| ID | Amino acid sequence/annotation |
|---|---|
| SEQ ID No: 1 | Human wild-type IL-15 protein |
| | NWVNVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| | QMFINTS |
| SEQ ID No: 2 | Human wild-type IL-15Rα protein fragment, IL-15 binding domain |
| | ITCPPPMSVEHADIWVKSYSLYSRERYICNSGFKRKAGTSSLTECVLNKATN |
| | VAHWTTPSLKCIR |
| SEQ ID No: 3 | 1st chain of the IgG1 Fc variant (V805) |
| | EPKSSDKTHTCPPCPAPELLRGPSVFLFPPKPKDTLYITREPEVTCVVVDVSH |
| | EDPEVKFNWYVDGVEVHNAKTKPREEQYNSTYRVVSVLTVLHQDWLNGK |
| | EYKCKVSNKARPAPIEKTISKAKGQPREPQVYTLPPCREEMTKNQVSLWCL |
| | VKGFYPSDIAVEWESNGQPENNYKTTPPVLDSDGSFFLYSKLTVDKSRWQQ |
| | GNVFSCSVMHEALHNHYTQKSLSLSPG |
| SEQ ID No: 4 | 2nd chain of the IgG1 Fc variant (V805) |
| | EPKSSDKTHTCPPCPAPELLRGPSVFLFPPKPKDTLYITREPEVTCVVVDVSH |
| | EDPEVKFNWYVDGVEVHNAKTKPREEQYNSTYRVVSVLTVLHQDWLNGK |
| | EYKCKVSNKARPAPIEKTISKAKGQPREPQVCTLPPSREEMTKNQVSLSCAV |
| | KGFYPSDIAVEWESNGQPENNYKTTPPVLDSDGSFFLVSKLTVDKSRWQQG |
| | NVFSCSVMHEALHNHYTQKSLSLSPG |
| SEQ ID No: 5 | 1st chain of the IgG4 Fc variant (V1) |
| | ESKYGPPCPPCPAPEFLGGPSVFLFPPKPKDTLMISRTPEVTCVVVDVSQEDP |
| | EVQFNWYVDGVEVHNAKTKPREEQFNSTYRVVSVLTVLHQDWLNGKEYK |
| | CKVSNKGLPSSIEKTISKAKGQPREPQVYTLPPCQEEMTKNQVSLWCLVKGF |
| | YPSDIAVEWESNGQPENNYKTTPPVLDSDGSFFLYSRLTVDKSRWQEGNVFS |
| | CSVMHEALHNHYTQKSLSLSLG |
| SEQ ID No: 6 | 2nd chain of the IgG4 Fc variant (V1) |
| | ESKYGPPCPPCPAPEFLGGPSVFLFPPKPKDTLMISRTPEVTCVVVDVSQEDP |
| | EVQFNWYVDGVEVHNAKTKPREEQFNSTYRVVSVLTVLHQDWLNGKEYK |
| | CKVSNKGLPSSIEKTISKAKGQPREPQVCTLPPSQEEMTKNQVSLSCAVKGF |
| | YPSDIAVEWESNGQPENNYKTTPPVLDSDGSFFLVSRLTVDKSRWQEGNVFS |
| | CSVMHEALHNHYTQKSLSLSLGK |
| SEQ ID No: 7 | 1st chain of the IgG4 Fc variant (V322) |
| | ESKYGPPCPPCPAPEAAGGPSVFLFPPKPKDTLMISRTPEVTCVVVDVSQEDP |
| | EVQFNWYVDGVEVHNAKTKPREEQFNSTYRVVSVLTVLHQDWLNGKEYK |
| | CKVSNKGLPSSIEKTISKAKGQPREPQVYTLPPCQEEMTKNQVSLWCLVKGF |
| | YPSDIAVEWESNGQPENNYKTTPPVLDSDGSFFLYSRLTVDKSRWQEGNVFS |
| | CSVMHEALHNHYTQKSLSLSLGK |
| SEQ ID No: 8 | 2nd chain of the IgG4 Fc variant (V322) |
| | ESKYGPPCPPCPAPEAAGGPSVFLFPPKPKDTLMISRTPEVTCVVVDVSQEDP |
| | EVQFNWYVDGVEVHNAKTKPREEQFNSTYRVVSVLTVLHQDWLNGKEYK |
| | CKVSNKGLPSSIEKTISKAKGQPREPQVCTLPPSQEEMTKNQVSLSCAVKGF |
| | YPSDIAVEWESNGQPENNYKTTPPVLDSDGSFFLVSRLTVDKSRWQEGNVFS |
| | CSVMHEALHNHYTQKSLSLSLGK |
| SEQ ID No: 9 | 1st chain of V0007.uIgG1V805 |
| | NWVNVISELKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| | QMFINTSEPKSSDKTHTCPPCPAPELLRGPSVFLFPPKPKDTLYITREPEVTCV |
| | VVDVSHEDPEVKFNWYVDGVEVHNAKTKPREEQYNSTYRVVSVLTVLHQ |
| | DWLNGKEYKCKVSNKARPAPIEKTISKAKGQPREPQVYTLPPCREEMTKNQ |
| | VSLWCLVKGFYPSDIAVEWESNGQPENNYKTTPPVLDSDGSFFLYSKLTVD |
| | KSRWQQGNVFSCSVMHEALHNHYTQKSLSLSPG |
| SEQ ID No: 10 | 2nd chain of V0007.uIgG1V805 |
| | ITCPPPMSVEHADIWVKSYSLYSRERYICNSGFKRKAGTSSLTECVLNKATN |
| | VAHWTTPSLKCIREPKSSDKTHTCPPCPAPELLRGPSVFLFPPKPKDTLYITRE |
| | PEVTCVVVDVSHEDPEVKFNWYVDGVEVHNAKTKPREEQYNSTYRVVSVL |
| | TVLHQDWLNGKEYKCKVSNKARPAPIEKTISKAKGQPREPQVCTLPPSREE |
| | MTKNQVSLSCAVKGFYPSDIAVEWESNGQPENNYKTTPPVLDSDGSFFLVS |
| | KLTVDKSRWQQGNVFSCSVMHEALHNHYTQKSLSLSPG |
| SEQ ID No: 11 | 1st Chain of V0008.uIgG4V1 |
| | NWVNVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| | QMFINTSESKYGPPCPPCPAPEFLGGPSVFLFPPKPKDTLMISRTPEVTCVVVD |
| | VSQEDPEVQFNWYVDGVEVHNAKTKPREEQFNSTYRVVSVLTVLHQDWLN |
| | GKEYKCKVSNKGLPSSIEKTISKAKGQPREPQVYTLPPCQEEMTKNQVSLWC |
| | LVKGFYPSDIAVEWESNGQPENNYKTTPPVLDSDGSFFLYSRLTVDKSRWQ |
| | EGNVFSCSVMHEALHNHYTQKSLSLSLG |
| SEQ ID No: 12 | 2nd chain of V0008.uIgG1V1 |
| | ITCPPPMSVEHADIWVKSYSLYSRERYICNSGFKRKAGTSSLTECVLNKATN |
| | VAHWTTPSLKCIRESKYGPPCPPCPAPEFLGGPSVFLFPPKPKDTLMISRTPEV |
| | TCVVVDVSQEDPEVQFNWYVDGVEVHNAKTKPREEQFNSTYRVVSVLTVL |
| | HQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLG |
| SEQ ID No: 13 | 1st chain of V0010.uIgG1V1 |
| | NWVNVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| | QMFINTESKYGPPCPPCPAPEFLGGPSVFLFPPKPKDTLMISRTPEVTCVVVD |
| | VSQEDPEVQFNWYVDGVEVHNAKTKPREEQFNSTYRVVSVLTVLHQDWLN |
| | GKEYKCKVSNKGLPSSIEKTISKAKGQPREPQVYTLPPCQEEMTKNQVSLWC |
| | LVKGFYPSDIAVEWESNGQPENNYKTTPPVLDSDGSFFLYSRLTVDKSRWQ |
| | EGNVFSCSVMHEALHNHYTQKSLSLSLG |
| SEQ ID No: 14 | 2nd chain of V0010.uIgG4V1 |
| | ITCPPPMSVEHADIWVKSYSLYSRERYICNSGFKRKAGTSSLTECVLNKATN |
| | VAHWTTPSLKCIRESKYGPPCPPCPAPEFLGGPSVFLFPPKPKDTLMISRTPEV |
| | TCVVVDVSQEDPEVQFNWYVDGVEVHNAKTKPREEQFNSTYRVVSVLTVL |
| | HQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLG |
| SEQ ID No: 15 | 1st chain of V0011.uIgG1V1 |
| | NWVNVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| | QMFINESKYGPPCPPCPAPEFLGGPSVFLFPPKPKDTLMISRTPEVTCVVVDV |
| | SQEDPEVQFNWYVDGVEVHNAKTKPREEQFNSTYRVVSVLTVLHQDWLNG |
| | KEYKCKVSNKGLPSSIEKTISKAKGQPREPQVYTLPPCQEEMTKNQVSLWCL |
| | VKGFYPSDIAVEWESNGQPENNYKTTPPVLDSDGSFFLYSRLTVDKSRWQE |
| | GNVFSCSVMHEALHNHYTQKSLSLSLG |
| SEQ ID No: 16 | 2nd chain of V0011.uIgG4V1 |
| | ITCPPPMSVEHADIWVKSYSLYSRERYICNSGFKRKAGTSSLTECVLNKATN |
| | VAHWTTPSLKCIRESKYGPPCPPCPAPEFLGGPSVFLFPPKPKDTLMISRTPEV |
| | TCVVVDVSQEDPEVQFNWYVDGVEVHNAKTKPREEQFNSTYRVVSVLTVL |
| | HQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLG |
| SEQ ID No: 17 | 1st chain of V0012.uIgG1V1 |
| | NWVNVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| | QMFIESKYGPPCPPCPAPEFLGGPSVFLFPPKPKDTLMISRTPEVTCVVVDVS |
| | QEDPEVQFNWYVDGVEVHNAKTKPREEQFNSTYRVVSVLTVLHQDWLNG |
| | KEYKCKVSNKGLPSSIEKTISKAKGQPREPQVYTLPPCQEEMTKNQVSLWCL |
| | VKGFYPSDIAVEWESNGQPENNYKTTPPVLDSDGSFFLYSRLTVDKSRWQE |
| | GNVFSCSVMHEALHNHYTQKSLSLSLG |
| SEQ ID No: 18 | 2nd chain of V0012.uIgG4V1 |
| | ITCPPPMSVEHADIWVKSYSLYSRERYICNSGFKRKAGTSSLTECVLNKATN |
| | VAHWTTPSLKCIRESKYGPPCPPCPAPEFLGGPSVFLFPPKPKDTLMISRTPEV |
| | TCVVVDVSQEDPEVQFNWYVDGVEVHNAKTKPREEQFNSTYRVVSVLTVL |
| | HQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLG |
| SEQ ID No: 19 | 1st chain of V0013.uIgG4V1 |
| | NWVNVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| | QMFESKYGPPCPPCPAPEFLGGPSVFLFPPKPKDTLMISRTPEVTCVVVDVSQ |
| | EDPEVQFNWYVDGVEVHNAKTKPREEQFNSTYRVVSVLTVLHQDWLNGK |
| | EYKCKVSNKGLPSSIEKTISKAKGQPREPQVYTLPPCQEEMTKNQVSLWCLV |
| | KGFYPSDIAVEWESNGQPENNYKTTPPVLDSDGSFFLYSRLTVDKSRWQEG |
| | NVFSCSVMHEALHNHYTQKSLSLSLG |
| SEQ ID No: 20 | 2nd chain of V0013.uIgG4V1 |
| | ITCPPPMSVEHADIWVKSYSLYSRERYICNSGFKRKAGTSSLTECVLNKATN |
| | VAHWTTPSLKCIRESKYGPPCPPCPAPEFLGGPSVFLFPPKPKDTLMISRTPEV |
| | TCVVVDVSQEDPEVQFNWYVDGVEVHNAKTKPREEQFNSTYRVVSVLTVL |
| | HQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLG |
| SEQ ID No: 21 | 1st chain of V0016.uIgG4V1 |
| | NWVNVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| | QMESKYGPPCPPCPAPEFLGGPSVFLFPPKPKDTLMISRTPEVTCVVVDVSQE |
| | DPEVQFNWYVDGVEVHNAKTKPREEQFNSTYRVVSVLTVLHQDWLNGKE |
| | YKCKVSNKGLPSSIEKTISKAKGQPREPQVYTLPPCQEEMTKNQVSLWCLVK |
| | GFYPSDIAVEWESNGQPENNYKTTPPVLDSDGSFFLYSRLTVDKSRWQEGN |
| | VFSCSVMHEALHNHYTQKSLSLSLG |
| SEQ ID No: 22 | 2nd chain of V0016.uIgG4V1 |
| | ITCPPPMSVEHADIWVKSYSLYSRERYICNSGFKRKAGTSSLTECVLNKATN |
| | VAHWTTPSLKCIRESKYGPPCPPCPAPEFLGGPSVFLFPPKPKDTLMISRTPEV |
| | TCVVVDVSQEDPEVQFNWYVDGVEVHNAKTKPREEQFNSTYRVVSVLTVL |
| | HQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLG |
| SEQ ID No: 23 | 1st chain of V0017.uIgG4V1 |
| | NWVNVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| | QESKYGPPCPPCPAPEFLGGPSVFLFPPKPKDTLMISRTPEVTCVVVDVSQED |
| | PEVQFNWYVDGVEVHNAKTKPREEQFNSTYRVVSVLTVLHQDWLNGKEY |
| | KCKVSNKGLPSSIEKTISKAKGQPREPQVYTLPPCQEEMTKNQVSLWCLVKG |
| | FYPSDIAVEWESNGQPENNYKTTPPVLDSDGSFFLYSRLTVDKSRWQEGNVF |
| | SCSVMHEALHNHYTQKSLSLSLG |
| SEQ ID No: 24 | 2nd chain of V0017.uIgG4V1 |
| | ITCPPPMSVEHADIWVKSYSLYSRERYICNSGFKRKAGTSSLTECVLNKATN |
| | VAHWTTPSLKCIRESKYGPPCPPCPAPEFLGGPSVFLFPPKPKDTLMISRTPEV |
| | TCVVVDVSQEDPEVQFNWYVDGVEVHNAKTKPREEQFNSTYRVVSVLTVL |
| | HQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLG |
| SEQ ID No: 25 | 1st chain of V0018.uIgG4V1 |
| | NWVNVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| | ESKYGPPCPPCPAPEFLGGPSVFLFPPKPKDTLMISRTPEVTCVVVDVSQEDP |
| | EVQFNWYVDGVEVHNAKTKPREEQFNSTYRVVSVLTVLHQDWLNGKEYK |
| | CKVSNKGLPSSIEKTISKAKGQPREPQVYTLPPCQEEMTKNQVSLWCLVKGF |
| | YPSDIAVEWESNGQPENNYKTTPPVLDSDGSFFLYSRLTVDKSRWQEGNVFS |
| | CSVMHEALHNHYTQKSLSLSLG |
| SEQ ID No: 26 | 2nd chain of V0018.uIgG4V1 |
| | ITCPPPMSVEHADIWVKSYSLYSRERYICNSGFKRKAGTSSLTECVLNKATN |
| | VAHWTTPSLKCIRESKYGPPCPPCPAPEFLGGPSVFLFPPKPKDTLMISRTPEV |
| | TCVVVDVSQEDPEVQFNWYVDGVEVHNAKTKPREEQFNSTYRVVSVLTVL |
| | HQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLG |
| SEQ ID No: 27 | 1st chain of V0019.uIgG4V1 |
| | WVNVISDLKKIEDLIQSMHIDATLYTESDVHPSCKVTAMKCFLLELQVISLES |
| | GDASIHDTVENLIILANNSLSSNGNVTESGCKECEELEEKNIKEFLQSFVHIVQ |
| | MFINTSESKYGPPCPPCPAPEFLGGPSVFLFPPKPKDTLMISRTPEVTCVVVDV |
| | SQEDPEVQFNWYVDGVEVHNAKTKPREEQFNSTYRVVSVLTVLHQDWLNG |
| | KEYKCKVSNKGLPSSIEKTISKAKGQPREPQVYTLPPCQEEMTKNQVSLWCL |
| | VKGFYPSDIAVEWESNGQPENNYKTTPPVLDSDGSFFLYSRLTVDKSRWQE |
| | GNVFSCSVMHEALHNHYTQKSLSLSLG |
| SEQ ID No: 28 | 2nd chain of V0019.uIgG4V1 |
| | ITCPPPMSVEHADIWVKSYSLYSRERYICNSGFKRKAGTSSLTECVLNKATN |
| | VAHWTTPSLKCIRESKYGPPCPPCPAPEFLGGPSVFLFPPKPKDTLMISRTPEV |
| | TCVVVDVSQEDPEVQFNWYVDGVEVHNAKTKPREEQFNSTYRVVSVLTVL |
| | HQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLG |
| SEQ ID No: 29 | 1st chain of V0020.uIgG4V1 |
| | VNVISDLKKIEDLIQSMHIDATLYTESDVHPSCKVTAMKCFLLELQVISLESG |
| | DASIHDTVENLIILANNSLSSNGNVTESGCKECEELEEKNIKEFLQSFVHIVQ |
| | MFINTSESKYGPPCPPCPAPEFLGGPSVFLFPPKPKDTLMISRTPEVTCVVVDV |
| | SQEDPEVQFNWYVDGVEVHNAKTKPREEQFNSTYRVVSVLTVLHQDWLNG |
| | KEYKCKVSNKGLPSSIEKTISKAKGQPREPQVYTLPPCQEEMTKNQVSLWCL |
| | VKGFYPSDIAVEWESNGQPENNYKTTPPVLDSDGSFFLYSRLTVDKSRWQE |
| | GNVFSCSVMHEALHNHYTQKSLSLSLG |
| SEQ ID No: 30 | 2nd chain of V0020.uIgG4V1 |
| | ITCPPPMSVEHADIWVKSYSLYSRERYICNSGFKRKAGTSSLTECVLNKATN |
| | VAHWTTPSLKCIRESKYGPPCPPCPAPEFLGGPSVFLFPPKPKDTLMISRTPEV |
| | TCVVVDVSQEDPEVQFNWYVDGVEVHNAKTKPREEQFNSTYRVVSVLTVL |
| | HQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLG |
| SEQ ID No: 31 | 1st chain of V0021.uIgG4V1 |
| | NVISDLKKIEDLIQSMHIDATLYTESDVHPSCKVTAMKCFLLELQVISLESGD |
| | ASIHDTVENLIILANNSLSSNGNVTESGCKECEELEEKNIKEFLQSFVHIVQMF |
| | INTSESKYGPPCPPCPAPEFLGGPSVFLFPPKPKDTLMISRTPEVTCVVVDVSQ |
| | EDPEVQFNWYVDGVEVHNAKTKPREEQFNSTYRVVSVLTVLHQDWLNGK |
| | EYKCKVSNKGLPSSIEKTISKAKGQPREPQVYTLPPCQEEMTKNQVSLWCLV |
| | KGFYPSDIAVEWESNGQPENNYKTTPPVLDSDGSFFLYSRLTVDKSRWQEG |
| | NVFSCSVMHEALHNHYTQKSLSLSLG |
| SEQ ID No: 32 | 2nd chain of V0021.uIgG4V1 |
| | ITCPPPMSVEHADIWVKSYSLYSRERYICNSGFKRKAGTSSLTECVLNKATN |
| | VAHWTTPSLKCIRESKYGPPCPPCPAPEFLGGPSVFLFPPKPKDTLMISRTPEV |
| | TCVVVDVSQEDPEVQFNWYVDGVEVHNAKTKPREEQFNSTYRVVSVLTVL |
| | HQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLG |
| SEQ ID No: 33 | 1st chain of V0022.uIgG4V1 |
| | VISDLKKIEDLIQSMHIDATLYTESDVHPSCKVTAMKCFLLELQVISLESGDA |
| | SIHDTVENLIILANNSLSSNGNVTESGCKECEELEEKNIKEFLQSFVHIVQMFI |
| | NTSESKYGPPCPPCPAPEFLGGPSVFLFPPKPKDTLMISRTPEVTCVVVDVSQ |
| | EDPEVQFNWYVDGVEVHNAKTKPREEQFNSTYRVVSVLTVLHQDWLNGK |
| | EYKCKVSNKGLPSSIEKTISKAKGQPREPQVYTLPPCQEEMTKNQVSLWCLV |
| | KGFYPSDIAVEWESNGQPENNYKTTPPVLDSDGSFFLYSRLTVDKSRWQEG |
| | NVFSCSVMHEALHNHYTQKSLSLSLG |
| SEQ ID No: 34 | 2nd chain of V0022.uIgG4V1 |
| | ITCPPPMSVEHADIWVKSYSLYSRERYICNSGFKRKAGTSSLTECVLNKATN |
| | VAHWTTPSLKCIRESKYGPPCPPCPAPEFLGGPSVFLFPPKPKDTLMISRTPEV |
| | TCVVVDVSQEDPEVQFNWYVDGVEVHNAKTKPREEQFNSTYRVVSVLTVL |
| | HQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLG |
| SEQ ID No: 35 | 1st chain of V0023.uIgG4V1 |
| | ISDLKKIEDLIQSMHIDATLYTESDVHPSCKVTAMKCFLLELQVISLESGDASI |
| | HDTVENLIILANNSLSSNGNVTESGCKECEELEEKNIKEFLQSFVHIVQMFINT |
| | SESKYGPPCPPCPAPEFLGGPSVFLFPPKPKDTLMISRTPEVTCVVVDVSQED |
| | PEVQFNWYVDGVEVHNAKTKPREEQFNSTYRVVSVLTVLHQDWLNGKEY |
| | KCKVSNKGLPSSIEKTISKAKGQPREPQVYTLPPCQEEMTKNQVSLWCLVKG |
| | FYPSDIAVEWESNGQPENNYKTTPPVLDSDGSFFLYSRLTVDKSRWQEGNVF |
| | SCSVMHEALHNHYTQKSLSLSLG |
| SEQ ID No: 36 | 2nd chain of V0023.uIgG4V1 |
| | ITCPPPMSVEHADIWVKSYSLYSRERYICNSGFKRKAGTSSLTECVLNKATN |
| | VAHWTTPSLKCIRESKYGPPCPPCPAPEFLGGPSVFLFPPKPKDTLMISRTPEV |
| | TCVVVDVSQEDPEVQFNWYVDGVEVHNAKTKPREEQFNSTYRVVSVLTVL |
| | HQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLG |
| SEQ ID No: 37 | 1st chain of V0024.uIgG4V1 |
| | SDLKKIEDLIQSMHIDATLYTESDVHPSCKVTAMKCFLLELQVISLESGDASI |
| | HDTVENLIILANNSLSSNGNVTESGCKECEELEEKNIKEFLQSFVHIVQMFINT |
| | SESKYGPPCPPCPAPEFLGGPSVFLFPPKPKDTLMISRTPEVTCVVVDVSQED |
| | PEVQFNWYVDGVEVHNAKTKPREEQFNSTYRVVSVLTVLHQDWLNGKEY |
| | KCKVSNKGLPSSIEKTISKAKGQPREPQVYTLPPCQEEMTKNQVSLWCLVKG |
| | FYPSDIAVEWESNGQPENNYKTTPPVLDSDGSFFLYSRLTVDKSRWQEGNVF |
| | SCSVMHEALHNHYTQKSLSLSLG |
| SEQ ID No: 38 | 2nd chain of V0024.uIgG4V1 |
| | ITCPPPMSVEHADIWVKSYSLYSRERYICNSGFKRKAGTSSLTECVLNKATN |
| | VAHWTTPSLKCIRESKYGPPCPPCPAPEFLGGPSVFLFPPKPKDTLMISRTPEV |
| | TCVVVDVSQEDPEVQFNWYVDGVEVHNAKTKPREEQFNSTYRVVSVLTVL |
| | HQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLG |
| SEQ ID No: 39 | 1st chain of V0025.uIgG4V1 |
| | DLKKIEDLIQSMHIDATLYTESDVHPSCKVTAMKCFLLELQVISLESGDASIH |
| | DTVENLIILANNSLSSNGNVTESGCKECEELEEKNIKEFLQSFVHIVQMFINTS |
| | ESKYGPPCPPCPAPEFLGGPSVFLFPPKPKDTLMISRTPEVTCVVVDVSQEDP |
| | EVQFNWYVDGVEVHNAKTKPREEQFNSTYRVVSVLTVLHQDWLNGKEYK |
| | CKVSNKGLPSSIEKTISKAKGQPREPQVYTLPPCQEEMTKNQVSLWCLVKGF |
| | YPSDIAVEWESNGQPENNYKTTPPVLDSDGSFFLYSRLTVDKSRWQEGNVFS |
| | CSVMHEALHNHYTQKSLSLSLG |
| SEQ ID No: 40 | 2nd chain of V0025.uIgG4V1 |
| | ITCPPPMSVEHADIWVKSYSLYSRERYICNSGFKRKAGTSSLTECVLNKATN |
| | VAHWTTPSLKCIRESKYGPPCPPCPAPEFLGGPSVFLFPPKPKDTLMISRTPEV |
| | TCVVVDVSQEDPEVQFNWYVDGVEVHNAKTKPREEQFNSTYRVVSVLTVL |
| | HQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLG |
| SEQ ID No: 41 | 1st chain of V0026.uIgG4V1 |
| | LKKIEDLIQSMHIDATLYTESDVHPSCKVTAMKCFLLELQVISLESGDASIHD |
| | TVENLIILANNSLSSNGNVTESGCKECEELEEKNIKEFLQSFVHIVQMFINTSE |
| | SKYGPPCPPCPAPEFLGGPSVFLFPPKPKDTLMISRTPEVTCVVVDVSQEDPE |
| | VQFNWYVDGVEVHNAKTKPREEQFNSTYRVVSVLTVLHQDWLNGKEYKC |
| | KVSNKGLPSSIEKTISKAKGQPREPQVYTLPPCQEEMTKNQVSLWCLVKGFY |
| | PSDIAVEWESNGQPENNYKTTPPVLDSDGSFFLYSRLTVDKSRWQEGNVFSC |
| | SVMHEALHNHYTQKSLSLSLG |
| SEQ ID No: 42 | 2nd chain of V0026.uIgG4V1 |
| | ITCPPPMSVEHADIWVKSYSLYSRERYICNSGFKRKAGTSSLTECVLNKATN |
| | VAHWTTPSLKCIRESKYGPPCPPCPAPEFLGGPSVFLFPPKPKDTLMISRTPEV |
| | TCVVVDVSQEDPEVQFNWYVDGVEVHNAKTKPREEQFNSTYRVVSVLTVL |
| | HQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLG |
| SEQ ID No: 43 | 1st chain of V0027.uIgG4V1 |
| | KKIEDLIQSMHIDATLYTESDVHPSCKVTAMKCFLLELQVISLESGDASIHDT |
| | VENLIILANNSLSSNGNVTESGCKECEELEEKNIKEFLQSFVHIVQMFINTSES |
| | KYGPPCPPCPAPEFLGGPSVFLFPPKPKDTLMISRTPEVTCVVVDVSQEDPEV |
| | QFNWYVDGVEVHNAKTKPREEQFNSTYRVVSVLTVLHQDWLNGKEYKCK |
| | VSNKGLPSSIEKTISKAKGQPREPQVYTLPPCQEEMTKNQVSLWCLVKGFYP |
| | SDIAVEWESNGQPENNYKTTPPVLDSDGSFFLYSRLTVDKSRWQEGNVFSCS |
| | VMHEALHNHYTQKSLSLSLG |
| SEQ ID No: 44 | 2nd chain of V0027.uIgG4V1 |
| | ITCPPPMSVEHADIWVKSYSLYSRERYICNSGFKRKAGTSSLTECVLNKATN |
| | VAHWTTPSLKCIRESKYGPPCPPCPAPEFLGGPSVFLFPPKPKDTLMISRTPEV |
| | TCVVVDVSQEDPEVQFNWYVDGVEVHNAKTKPREEQFNSTYRVVSVLTVL |
| | HQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLG |
| SEQ ID No: 45 | 1st chain of V0028.uIgG4V1 |
| | KIEDLIQSMHIDATLYTESDVHPSCKVTAMKCFLLELQVISLESGDASIHDTV |
| | ENLIILANNSLSSNGNVTESGCKECEELEEKNIKEFLQSFVHIVQMFINTSESK |
| | YGPPCPPCPAPEFLGGPSVFLFPPKPKDTLMISRTPEVTCVVVDVSQEDPEVQ |
| | FNWYVDGVEVHNAKTKPREEQFNSTYRVVSVLTVLHQDWLNGKEYKCKV |
| | SNKGLPSSIEKTISKAKGQPREPQVYTLPPCQEEMTKNQVSLWCLVKGFYPS |
| | DIAVEWESNGQPENNYKTTPPVLDSDGSFFLYSRLTVDKSRWQEGNVFSCS |
| | VMHEALHNHYTQKSLSLSLG |
| SEQ ID No: 46 | 2nd chain of V0028.uIgG4V1 |
| | ITCPPPMSVEHADIWVKSYSLYSRERYICNSGFKRKAGTSSLTECVLNKATN |
| | VAHWTTPSLKCIRESKYGPPCPPCPAPEFLGGPSVFLFPPKPKDTLMISRTPEV |
| | TCVVVDVSQEDPEVQFNWYVDGVEVHNAKTKPREEQFNSTYRVVSVLTVL |
| | HQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLG |
| SEQ ID No: 47 | 1st chain of V0029.uIgG4V1 |
| | IEDLIQSMHIDATLYTESDVHPSCKVTAMKCFLLELQVISLESGDASIHDTVE |
| | NLIILANNSLSSNGNVTESGCKECEELEEKNIKEFLQSFVHIVQMFINTSESKY |
| | GPPCPPCPAPEFLGGPSVFLFPPKPKDTLMISRTPEVTCVVVDVSQEDPEVQF |
| | NWYVDGVEVHNAKTKPREEQFNSTYRVVSVLTVLHQDWLNGKEYKCKVS |
| | NKGLPSSIEKTISKAKGQPREPQVYTLPPCQEEMTKNQVSLWCLVKGFYPSD |
| | IAVEWESNGQPENNYKTTPPVLDSDGSFFLYSRLTVDKSRWQEGNVFSCSV |
| | MHEALHNHYTQKSLSLSLG |
| SEQ ID No: 48 | 2nd chain of V0029.uIgG4V1 |
| | ITCPPPMSVEHADIWVKSYSLYSRERYICNSGFKRKAGTSSLTECVLNKATN |
| | VAHWTTPSLKCIRESKYGPPCPPCPAPEFLGGPSVFLFPPKPKDTLMISRTPEV |
| | TCVVVDVSQEDPEVQFNWYVDGVEVHNAKTKPREEQFNSTYRVVSVLTVL |
| | HQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLG |
| SEQ ID No: 49 | 1st chain of V0030.uIgG4V1 |
| | EDLIQSMHIDATLYTESDVHPSCKVTAMKCFLLELQVISLESGDASIHDTVEN |
| | LIILANNSLSSNGNVTESGCKECEELEEKNIKEFLQSFVHIVQMFINTSESKYG |
| | PPCPPCPAPEFLGGPSVFLFPPKPKDTLMISRTPEVTCVVVDVSQEDPEVQFN |
| | WYVDGVEVHNAKTKPREEQFNSTYRVVSVLTVLHQDWLNGKEYKCKVSN |
| | KGLPSSIEKTISKAKGQPREPQVYTLPPCQEEMTKNQVSLWCLVKGFYPSDI |
| | AVEWESNGQPENNYKTTPPVLDSDGSFFLYSRLTVDKSRWQEGNVFSCSVM |
| | HEALHNHYTQKSLSLSLG |
| SEQ ID No: 50 | 2nd chain of V0030.uIgG4V1 |
| | ITCPPPMSVEHADIWVKSYSLYSRERYICNSGFKRKAGTSSLTECVLNKATN |
| | VAHWTTPSLKCIRESKYGPPCPPCPAPEFLGGPSVFLFPPKPKDTLMISRTPEV |
| | TCVVVDVSQEDPEVQFNWYVDGVEVHNAKTKPREEQFNSTYRVVSVLTVL |
| | HQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLG |
| SEQ ID No: 51 | IL-15 variant comprised in V0013.uIgG4V1 |
| | NWVNVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| | QMF |
| SEQ ID No: 52 | IL-15 variant comprised in V0016.uIgG4V1 |
| | NWVNVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| | QM |
| SEQ ID No: 53 | IL-15 variant comprised in V0017.uIgG4V1 |
| | NWVNVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| | Q |
| SEQ ID No: 54 | IL-15 variant comprised in V0018.uIgG4V1 |
| | NWVNVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| SEQ ID No: 55 | IL-15 variant comprised in V0010.uIgG4V1 |
| | NWVNVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| | QMFINT |
| SEQ ID No: 56 | IL-15 variant comprised in V0011.uIgG4V1 |
| | NWVNVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| | QMFIN |
| SEQ ID No: 57 | IL-15 variant comprised in V0012.uIgG4V1 |
| | NWVNVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| | QMFI |
| SEQ ID No: 58 | IL-15 variant comprised in V0019.uIgG4V1 |
| | WVNVISDLKKIEDLIQSMHIDATLYTESDVHPSCKVTAMKCFLLELQVISLES |
| | GDASIHDTVENLIILANNSLSSNGNVTESGCKECEELEEKNIKEFLQSFVHIVQ |
| | MFINTS |
| SEQ ID No: 59 | IL-15 variant comprised in V0020.uIgG4V1 |
| | VNVISDLKKIEDLIQSMHIDATLYTESDVHPSCKVTAMKCFLLELQVISLESG |
| | DASIHDTVENLIILANNSLSSNGNVTESGCKECEELEEKNIKEFLQSFVHIVQ |
| | MFINTS |
| SEQ ID No: 60 | IL-15 variant comprised in V0021.uIgG4V1 |
| | NVISDLKKIEDLIQSMHIDATLYTESDVHPSCKVTAMKCFLLELQVISLESGD |
| | ASIHDTVENLIILANNSLSSNGNVTESGCKECEELEEKNIKEFLQSFVHIVQMF |
| | INTS |
| SEQ ID No: 61 | IL-15 variant comprised in V0022.uIgG4V1 |
| | VISDLKKIEDLIQSMHIDATLYTESDVHPSCKVTAMKCFLLELQVISLESGDA |
| | SIHDTVENLIILANNSLSSNGNVTESGCKECEELEEKNIKEFLQSFVHIVQMFI |
| | NTS |
| SEQ ID No: 62 | IL-15 variant comprised in V0023.uIgG4V1 |
| | ISDLKKIEDLIQSMHIDATLYTESDVHPSCKVTAMKCFLLELQVISLESGDASI |
| | HDTVENLIILANNSLSSNGNVTESGCKECEELEEKNIKEFLQSFVHIVQMFINT |
| | S |
| SEQ ID No: 63 | IL-15 variant comprised in V0024.uIgG4V1 |
| | SDLKKIEDLIQSMHIDATLYTESDVHPSCKVTAMKCFLLELQVISLESGDASI |
| | HDTVENLIILANNSLSSNGNVTESGCKECEELEEKNIKEFLQSFVHIVQMFINT |
| | S |
| SEQ ID No: 64 | IL-15 variant comprised in V0025.uIgG4V1 |
| | DLKKIEDLIQSMHIDATLYTESDVHPSCKVTAMKCFLLELQVISLESGDASIH |
| | DTVENLIILANNSLSSNGNVTESGCKECEELEEKNIKEFLQSFVHIVQMFINTS |
| SEQ ID No: 65 | IL-15 variant comprised in V0026.uIgG4V1 |
| | LKKIEDLIQSMHIDATLYTESDVHPSCKVTAMKCFLLELQVISLESGDASIHD |
| | TVENLIILANNSLSSNGNVTESGCKECEELEEKNIKEFLQSFVHIVQMFINTS |
| SEQ ID No: 66 | IL-15 variant comprised in V0027.uIgG4V1 |
| | KKIEDLIQSMHIDATLYTESDVHPSCKVTAMKCFLLELQVISLESGDASIHDT |
| | VENLIILANNSLSSNGNVTESGCKECEELEEKNIKEFLQSFVHIVQMFINTS |
| SEQ ID No: 67 | IL-15 variant comprised in V0028.uIgG4V1 |
| | KIEDLIQSMHIDATLYTESDVHPSCKVTAMKCFLLELQVISLESGDASIHDTV |
| | ENLIILANNSLSSNGNVTESGCKECEELEEKNIKEFLQSFVHIVQMFINTS |
| SEQ ID No: 68 | IL-15 variant comprised in V0029.uIgG4V1 |
| | IEDLIQSMHIDATLYTESDVHPSCKVTAMKCFLLELQVISLESGDASIHDTVE |
| | NLIILANNSLSSNGNVTESGCKECEELEEKNIKEFLQSFVHIVQMFINTS |
| SEQ ID No: 69 | IL-15 variant comprised in V0030.uIgG4V1 |
| | EDLIQSMHIDATLYTESDVHPSCKVTAMKCFLLELQVISLESGDASIHDTVEN |
| | LIILANNSLSSNGNVTESGCKECEELEEKNIKEFLQSFVHIVQMFINTS |
| SEQ ID No: 70 | 1st chain of V0013.uIgG4V322 |
| | NWVNVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| | QMFESKYGPPCPPCPAPEAAGGPSVFLFPPKPKDTLMISRTPEVTCVVVDVS |
| | QEDPEVQFNWYVDGVEVHNAKTKPREEQFNSTYRVVSVLTVLHQDWLNG |
| | KEYKCKVSNKGLPSSIEKTISKAKGQPREPQVYTLPPCQEEMTKNQVSLWCL |
| | VKGFYPSDIAVEWESNGQPENNYKTTPPVLDSDGSFFLYSRLTVDKSRWQE |
| | GNVFSCSVMHEALHNHYTQKSLSLSLGK |
| SEQ ID No: 71 | 2nd chain of V0013.uIgG4V322 |
| | ITCPPPMSVEHADIWVKSYSLYSRERYICNSGFKRKAGTSSLTECVLNKATN |
| | VAHWTTPSLKCIRESKYGPPCPPCPAPEAAGGPSVFLFPPKPKDTLMISRTPE |
| | VTCVVVDVSQEDPEVQFNWYVDGVEVHNAKTKPREEQFNSTYRVVSVLTV |
| | LHQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLGK |
| SEQ ID No: 72 | 1st chain of V0013.uIgG4V803 |
| | NWVNVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| | QMFESKYGPPCPPCPAPEAAGGPSVFLFPPKPKDTLYITREPEVTCVVVDVSQ |
| | EDPEVQFNWYVDGVEVHNAKTKPREEQFNSTYRVVSVLTVLHQDWLNGK |
| | EYKCKVSNKGLPSSIEKTISKAKGQPREPQVYTLPPCQEEMTKNQVSLWCLV |
| | KGFYPSDIAVEWESNGQPENNYKTTPPVLDSDGSFFLYSRLTVDKSRWQEG |
| | NVFSCSVMHEALHNHYTQKSLSLSLGK |
| SEQ ID No: 73 | 2nd chain of V0013.uIgG4V803 |
| | ITCPPPMSVEHADIWVKSYSLYSRERYICNSGFKRKAGTSSLTECVLNKATN |
| | VAHWTTPSLKCIRESKYGPPCPPCPAPEAAGGPSVFLFPPKPKDTLYITREPE |
| | VTCVVVDVSQEDPEVQFNWYVDGVEVHNAKTKPREEQFNSTYRVVSVLTV |
| | LHQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLGK |
| SEQ ID No: 74 | 1st chain of V0013.uIgG4V805 |
| | NWVNVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| | QMFEPKSSDKTHTCPPCPAPELLRGPSVFLFPPKPKDTLYITREPEVTCVVVD |
| | VSHEDPEVKFNWYVDGVEVHNAKTKPREEQYNSTYRVVSVLTVLHQDWL |
| | NGKEYKCKVSNKARPAPIEKTISKAKGQPREPQVYTLPPCREEMTKNQVSL |
| | WCLVKGFYPSDIAVEWESNGQPENNYKTTPPVLDSDGSFFLYSKLTVDKSR |
| | WQQGNVFSCSVMHEALHNHYTQKSLSLSPG |
| SEQ ID No: 75 | 2nd chain of V0013.uIgG4V805 |
| | ITCPPPMSVEHADIWVKSYSLYSRERYICNSGFKRKAGTSSLTECVLNKATN |
| | VAHWTTPSLKCIREPKSSDKTHTCPPCPAPELLRGPSVFLFPPKPKDTLYITRE |
| | PEVTCVVVDVSHEDPEVKFNWYVDGVEVHNAKTKPREEQYNSTYRVVSVL |
| | TVLHQDWLNGKEYKCKVSNKARPAPIEKTISKAKGQPREPQVCTLPPSREE |
| | MTKNQVSLSCAVKGFYPSDIAVEWESNGQPENNYKTTPPVLDSDGSFFLVS |
| | KLTVDKSRWQQGNVFSCSVMHEALHNHYTQKSLSLSPG |
| SEQ ID No: 76 | 1st chain of W327199-T1U2.P13-1.uIgG4V322 |
| | NWVNVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSFVHIV |
| | QMFESKYGPPCPPCPAPEAAGGPSVFLFPPKPKDTLMISRTPEVTCVVVDVS |
| | QEDPEVQFNWYVDGVEVHNAKTKPREEQFNSTYRVVSVLTVLHQDWLNG |
| | KEYKCKVSNKGLPSSIEKTISKAKGQPREPQVYTLPPCQEEMTKNQVSLWCL |
| | VKGFYPSDIAVEWESNGQPENNYKTTPPVLDSDGSFFLYSRLTVDKSRWQE |
| | GNVFSCSVMHEALHNHYTQKSLSLSLGGGGGSGGGGSGGGGSGGGGSGGG |
| | GSGGGGSGGGGSQVQLVESGGGVVQPGGSLRLSCAASDSIDSLVNMGWYR |
| | QAPGKQRELVALIATYITHYADFVKGRFTISRDNSKNTLYLQMNSLRAEDTA |
| | VYYCYARNIIVDYWGQGTLVTVS |
| SEQ ID No: 77 | 2nd chain of W327199-T1U2.P13-1.uIgG4V322 |
| | ITCPPPMSVEHADIWVKSYSLYSRERYICNSGFKRKAGTSSLTECVLNKATN |
| | VAHWTTPSLKCIRESKYGPPCPPCPAPEAAGGPSVFLFPPKPKDTLMISRTPE |
| | VTCVVVDVSQEDPEVQFNWYVDGVEVHNAKTKPREEQFNSTYRVVSVLTV |
| | LHQDWLNGKEYKCKVSNKGLPSSIEKTISKAKGQPREPQVCTLPPSQEEMTK |
| | NQVSLSCAVKGFYPSDIAVEWESNGQPENNYKTTPPVLDSDGSFFLVSRLTV |
| | DKSRWQEGNVFSCSVMHEALHNHYTQKSLSLSLGGGGGSGGGGSGGGGSG |
| | GGGSGGGGSGGGGSGGGGSQVQLVESGGGVVQPGGSLRLSCAASDSIDSLV |
| | NMGWYRQAPGKQRELVALIATYITHYADFVKGRFTISRDNSKNTLYLQMNS |
| | LRAEDTAVYYCYARNIIVDYWGQGTLVTVS |
| SEQ ID No: 78 | IL-2/15Rβ (CD122) ECD |
| | AVNGTSQFTCFYNSRANISCVWSQDGALQDTSCQVHAWPDRRRWNQTCEL |
| | LPVSQASWACNLILGAPDSQKLTTVDIVTLRVLCREGVRWRVMAIQDFKPF |
| | ENLRLMAPISLQVVHVETHRCNISWEISQASHYFERHLEFEARTLSPGHTWE |
| | EAPLLTLKQKQEWICLETLTPDTQYEFQVRVKPLQGEFTTWSPWSQPLAFRT |
| | KPAALGKDLEGGGGSGGGGHHHHHH |
| SEQ ID No: 79 | Common γ chain (CD131) ECD |
| | LNTTILTPNGNEDTTADFFLTTMPTDSLSVSTLPLPEVQCFVFNVEYMNCTW |
| | NSSSEPQPTNLTLHYWYKNSDNDKVQKCSHYLFSEEITSGCQLQKKEIHLYQ |
| | TFVVQLQDPREPRRQATQMLKLQNLVIPWAPENLTLHKLSESQLELNWNNR |
| | FLNHCLEHLVQYRTDWDHSWTEQSVDYRHKFSLPSVDGQKRYTFRVRSRF |
| | NPLCGSAQHWSEWSHPIHWGSNTSKENLEGGGGSGGGGHHHHHH |
| SEQ ID No: 80 | anti-PD-1 VHH |
| | QVQLVESGGGVVQPGGSLRLSCAASDSIDSLVNMGWYRQAPGKQRELVALI |
| | ATYITHYADFVKGRFTISRDNSKNTLYLQMNSLRAEDTAVYYCYARNIIVDY |
| | WGQGTLVTVS |

### EXAMPLES

The present disclosure, thus generally described, will be understood more readily by reference to the following Examples, which are provided by way of illustration and are not intended to be limiting of the present disclosure. The Examples are not intended to represent that the experiments below are all or the only experiments performed.

### Example 1

### Preparation of Materials, IL-15 Variants and Fusion Proteins

### 1.1 Preparation of Materials

Information on the commercially available materials used in the examples is provided in Table 1.

TABLE 1

| Materials | Vendor | Cat. |
|---|---|---|
| Expi293 Expression system | Thermo Fisher | A14635 |
| ExpiFectamine293 transfection kit | Thermo Fisher | A14524 |
| Expi293F ™ expression medium | Thermo Fisher | A1435101 |
| Lipofectamine ™ 2000 Transfection Reagent | Thermo Fisher | 11668019 |
| FreeStyle ™ 293-F Cells | Thermo Fisher | R79007 |
| FreeStyle ™ 293 Expression Medium | Thermo Fisher | 12338002 |
| Opti-MEM | Thermo Fisher | 31985070 |
| Ni column | GE healthcare | 173712 |
| Protein A column | GE healthcare | 175438 |
| Superdex200 prep grade | GE Healthcare | 17-1043-01 |
| HPLC-SEC | TOSOH | 0008541 |
| NuPAGE4%-12% Bis-Tris Gel | Thermo Fisher | NP0322BOX |
| Mouse anti-human CD3-FITC | BioLegend | 317306 |
| APC-Cy7 Mouse Anti-Human CD8 | BD Biosciences | 557834 |
| Mouse Anti-Human CD56-PE | BioLegend | 318306 |
| APC anti-human Ki-67 Antibody | BioLegend | 350514 |
| FITC Mouse anti-human CD3e | Invitrogen | MA1-10178 |
| APC Mouse Anti-Human CD16 clone 3G8 | BD Pharmingen | 561248 |
| PE-Cy7 Mouse anti-Ki67 clone B56 | BD Pharmingen | 581283 |
| FITC anti-mouse CD3 Antibody | BioLegend | 100204 |
| APC-Cy7 rat anti-mouse CD8a | BD Biosciences | 557654 |
| PE Rat anti-mouse CD335 | BD Pharmingen | 560757 |
| APC anti-mouse Ki-67 Antibody | BioLegend | 652406 |
| Fixation/Permeabilization concentrate | Invitrogen | 00-5123-43 |
| Fixation/Perm Diluent | Invitrogen | 00-5223-56 |
| Permeabilization Buffer 10X | Invitrogen | 00-8333-56 |
| Ficoll | Stem Cell | 7861 |
| ACK Lysing Buffer | GIBCO | A10492-01 |
| RPMI 1640 Medium | Gibco | A1049101 |
| Fetal Bovine Serum | ExCell Bio | FND500 |
| Penicillin-Streptomycin Solution | Hyclone | SV30010 |
| 2-mercaptoethanol | Sigma | M6250 |
| Lymphoprep | STEMCELL | 07851 |
| DPBS | Corning | 21-031-CVR |
| 96 Well Microplates | Corning | 3799 |
| Goat Anti-Human IgG | Southern Biotech | 2049-01 |
| Goat Anti-human IgG-Fc-biotin | Southern Biotech | SB-2049-08 |
| Human IL-15 biotinylated antibody | R&D | BAM247 |
| Goat anti-mouse IgG Fc fragment cross- | BETHYL | A90-231P |
| adsorbed HRP | | |
| Streptavidin-HRP | Life Science | Life-SNN1004 |
| TMB | Solarbio | PR1200 |

### 1.2 Generation of IL-15 Variants, Fusion Proteins and BMKs

### Construction of Expression Vectors for BMKs, IL15/IL15Rα-Fc Variants and PD-1/IL-15 Variant Fusion Protein

Two heterodimeric IL15/IL15Rα-Fc fusion proteins, XENP24306 and XENP20818, disclosed in US20180118805A1 were used as benchmarks (referred to herein as “W327151-BMK1” and “W327151-BMK2”, respectively) in the following experiments. XENP24306 utilizes an IL-15 variant comprising D30N/E64Q/N65D mutation while XENP20818 utilizes a wild-type IL-15 protein. The DNA segments of XENP24306 and XENP20818 were synthesized according to US20180118805A1 (Seq.382-387 and Seq.52-57) and sub-cloned into pcDNA3.4 expression vectors, then used in transfection for protein production.

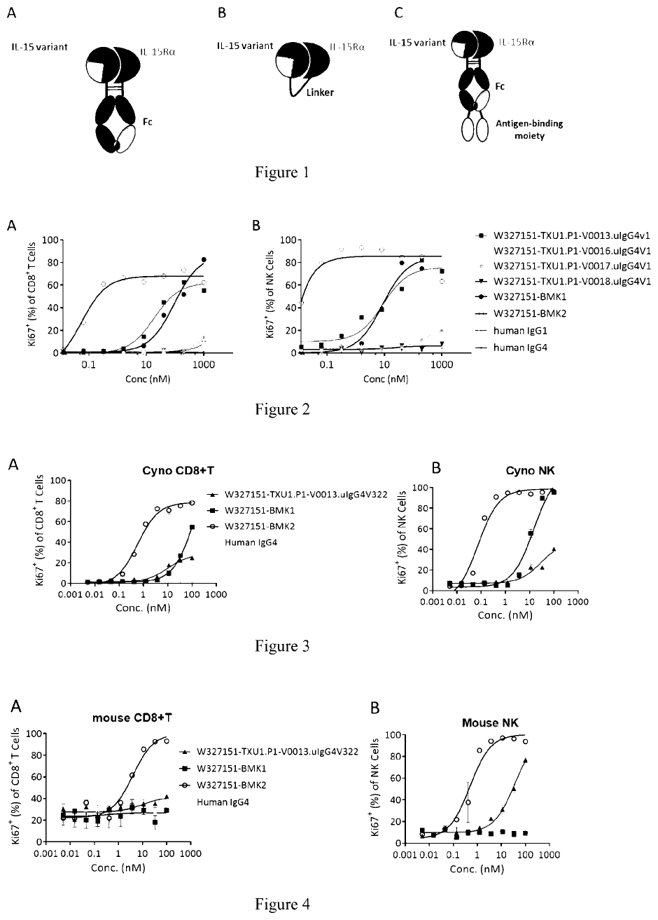

DNA fragments encoding the IL-15 variants listed in Table 2, IL15Rα, IgG4 V1 format, IgG4 V322 format, IgG4 V803 format, and IgG1V805 format were also synthesized, sub-cloned into separate expression vectors and transfected for protein production. Compared to human wild-type IgG Fc, IgG4 V1 format comprises S228P, IgG4 V322 format comprises S228P and “FALA” (F234A/L235A), IgG4 V803 format comprises S228P, “FALA” and “YTE”, while IgG1 V805 format comprises “YTE” (M252Y/S254T/T256E) and “RR” (G236R/L328R). The structure of the assembled polypeptide complexes is as shown in FIG. 1A. For simplicity, the naming of these heterodimeric IL-15/IL15Rα polypeptide complexes may also be abbreviated in the format “V00xx.uIgG4v1”, “V00xx.uIgG4v322”, “V00xx.uIgG4v803” or “V00xx.uIgG1v805” throughout the specification, in which “V00xx” indicates the numbering of the IL-15 variant.

Further, antibodies comprising the heterodimeric IL15/IL15Rα-Fc fusion protein as described above fused to a previously developed anti-PD-1 VHH (sequence as shown in SEQ ID No: 80) were constructed, and designated as “W327199-T1U1.P1-13.uIgG4V322”. DNA fragments encoding the PD-1/IL-15 variants in IgG4V322 format listed in Table A (SEQ ID No: 76-77) were synthesized. The structure of the assembled polypeptide complexes is as shown in FIG. 1C, wherein IL-15 and IL15Rα are at the N terminal and the PD-1 binding moiety is at the C terminal, intervened by the Fc region.

TABLE 2
IL-15/IL15Rα heterodimeric polypeptide complexes

| Protein ID | Mutations on IL15 |
|---|---|
| W327151-TXU1.P1-V0007.uIgG1v805 | WT without linker (IgG1 Fc) |
| W327151-TXU1.P1-V0008.uIgG4v1 | WT without linker (IgG4 Fc) |
| W327151-TXU1.P1-V0010.uIgG4v1 | Delete C terminal S114 |
| W327151-TXU1.P1-V0011.uIgG4v1 | Delete C terminal T113, S114 |
| W327151-TXU1.P1-V0012.uIgG4v1 | Delete C terminal N112, T113, S114 |
| W327151-TXU1.P1-V0013.uIgG4v1 | Delete C terminal I111, N112, T113, S114 |
| W327151-TXU1.P1-V0016.uIgG4v1 | Delete C terminal F110, I111, N112, T113, |
| | S114 |
| W327151-TXU1.P1-V0017.uIgG4v1 | Delete C terminal M109, F110, I111, N112, |
| | T113, S114 |
| W327151-TXU1.P1-V0018.uIgG4v1 | Delete C terminal Q108, M109, F110, I111, |
| | N112, T113, S114 |
| W327151-TXU1.P1-V0019.uIgG4v1 | Delete N terminal N1 |
| W327151-TXU1.P1-V0020.uIgG4v1 | Delete N terminal N1, W2 |
| W327151-TXU1.P1-V0021.uIgG4v1 | Delete N terminal N1, W2, V3 |
| W327151-TXU1.P1-V0022.uIgG4v1 | Delete N terminal N1, W2, V3, N4 |
| W327151-TXU1.P1-V0023.uIgG4v1 | Delete N terminal N1, W2, V3, N4, V5 |
| W327151-TXU1.P1-V0024.uIgG4v1 | Delete N terminal N1, W2, V3, N4, V5, I6 |
| W327151-TXU1.P1-V0025.uIgG4v1 | Delete N terminal N1, W2, V3, N4, V5, I6, |
| | S7 |
| W327151-TXU1.P1-V0026.uIgG4v1 | Delete N terminal N1, W2, V3, N4, V5, I6, |
| | S7, D8 |
| W327151-TXU1.P1-V0027.uIgG4v1 | Delete N terminal N1, W2, V3, N4, V5, I6, |
| | S7, D8, L9 |
| W327151-TXU1.P1-V0028.uIgG4v1 | Delete N terminal N1, W2, V3, N4, V5, I6, |
| | S7, D8, L9, K10 |
| W327151-TXU1.P1-V0029.uIgG4v1 | Delete N terminal N1, W2, V3, N4, V5, I6, |
| | S7, D8, L9, K10, K11 |
| W327151-TXU1.P1-V0030.uIgG4v1 | Delete N terminal N1, W2, V3, N4, V5, I6, |
| | S7, D8, L9, K10, K11, I12 |

### Small Scale Protein Expression in HEK 293 Cells

Expi293 cells (Thermofisher, A14635) were transfected with plasmids constructed as described above, cultured for 5 days following the manufacturer suggested protocol. The supernatants were collected and analyzed by SDS-PAGE, or filtered and used for target protein purification using Protein A column (GE Healthcare, Cat. 175438). The concentration of purified Fc-tagged proteins was determined by absorbance at 280 nm. The size and purity were tested by SDS-PAGE and SEC-HPLC, respectively, and then stored at −80° C.

### Purity by SEC-HPLC

The purity of proteins was tested by SEC-HPLC using Agilent 1260 Infinity HPLC. 50 μL of antibody solution was injected on a TSKgel SuperSW3000 column using 50 mM sodium phosphate, 0.15 M NaCl, pH 7.0 buffer. The running time was 20 min. Peak retention times on the column were monitored at 280 nm. Data was analyzed using ChemStation software (V2.99.2.0).

### Example 2

### In Vitro Characterization

### 2.1 Differential Scanning Fluorimetry (DSF)

A DSF assay was performed using 7500 Fast Real-Time PCR system (Applied Biosystems). Briefly, 19 μL of protein solution was mixed with 1 μl of 62.5×SYPRO Orange solution (Thermofisher-S6650) and added to a 96 well plate. The plate was heated from 26° C. to 95° C. at a rate of 2° C./min and the resulting fluorescence data was collected. The data was analyzed automatically by its operation software and Tm was calculated by taking the maximal value of negative derivative of the resulting fluorescence data with respect to temperature. Ton can be roughly determined as the temperature of negative derivative plot beginning to decrease from a pre-transition baseline.

The thermo-stability of IL-15 variants was shown in Table 3, which indicates that mutagenesis via amino acid deletion had no impact on thermo-stability of IL-15.

TABLE 3
DSF result of IL-15 heterodimer fusion proteins.

| Protein Name | Tm1 (° C.) | Tm2 (° C.) |
|---|---|---|
| W327151-TXU1.P1-V0013.uIgG4v1 | 61.4 | — |
| W327151-TXU1.P1-V0016.uIgG4V1 | 60.1 | — |
| W327151-TXU1.P1-V0017.uIgG4V1 | 60.5 | — |
| W327151-TXU1.P1-V0018.uIgG4V1 | 59.3 | 62.6 |
| W327151-BMK1 | 61.3 | 79.0 |
| W327151-BMK2 | 65.5 | — |

### 2.2 Thermal Stability Test by DLS-Tagg

Tagg onset measurement was investigated using DynaPro Plate Reader III (Wyatt Dynapro™). Sample in 7.5 μL was mixed with 5 μL silicone oil in microplate. The plate was heated from 40° C. to 80° C. at a rate of 0.125° C./min. 3 acquisitions were collected for each protein sample while each acquisition time was 5 s. For each measurement, the diffusion coefficient was determined and plotted against temperature. Tagg onset values were calculated automatically by the operation software (DYNAMICS 7.8.1.3). As shown in Table 4, V0013 showed good thermal stability.

TABLE 4
DLS-Tagg result of IL-15 heterodimer fusion protein.

| Protein Name | Tagg onset (° C.) |
|---|---|
| W327151-TXU1.P1-V0013.uIgG4V322 | 67.3 |

### 2.3 Determination of Diffusion Interaction Parameter (kD) by DLS

The kD measurement was investigated using DynaPro Plate Reader III (Wyatt Dynapro™). Samples were first diluted to a final concentration at 2.5, 5, 10, 15, and 20 mg/mL in microplate. Data collection was performed by the DYNAMICS operation software (v7.8.1.3). 5 acquisitions were collected for each protein sample while each acquisition time was 5 s. For each measurement, the diffusion coefficient was determined and plotted against protein concentration. kD values were calculated automatically by the software. The kD value showed in Table 5 was in normal range, meaning low aggregation risk of V0013.

TABLE 5
DLS-kD result of IL-15 heterodimer fusion protein.

| Protein Name | kD (mL/g) | Size Distribution |
|---|---|---|
| W327151-TXU1.P1-V0013.uIgG4V322 | −0.6 | monodisperse size |

### 2.4 Determination of Self-Interaction by AC-SINS

Self-Interaction of antibodies was investigated using AC-SINS method. Goat anti-human IgG Fc antibodies (capture) and ChromPure Goat IgG antibodies (non-capture) were mixed previously, then the IgG mixture was mixed with gold nanoparticle (AuNP) solution at a volume ratio of 1:9. All antibodies in 0.1 mg/mL was mixed with AuNP solution followed by incubation. Antibody-AuNP mixture was detected by absorbance value from 510 to 570 nm. Δλmax value was calculated by subtraction the max absorbance value of samples with that of buffer. The Δλmax of V0013 was −3 nm (Table 6), indicating no self-interaction risk.

TABLE 6
AC-SINS result of IL-15 heterodimer fusion protein.

| Protein Name | Δ λ max (nm) |
|---|---|
| W327151-TXU1.P1-V0013.uIgG4V322 | −3 |

### 2.5 Freeze/Thaw Stress Test

Each sample in equivalent concentration was frozen at −80° C. and thawed at 25° C. for an hour respectively. After 5 repeated freeze/thaw cycling, the samples before and after the treatment were tested by concentration and SEC-HPLC. After 5 times frozen and thawed (5× F/T), V0013 was stable in concentration and purity by SEC-HPLC (Table 7), nearly no more aggregation. The appearance of V0013 was better than BMK1.

TABLE 7
Freeze/thaw stress test result of IL-15 heterodimer fusion protein.

| | | W327151-BMK1 | | | W327151-TXU1.P1-V0013.ulgG4V322 | | |
|---|---|---|---|---|---|---|---|
| Design (−80° C.&RT) | | HMW % | Mono % | LMW % | HMW % | Mono % | LMW % |
| SEC-HPLC | F/T T0 | 6.56 | 93.44 | 0 | 2.13 | 97.87 | 0 |
| | F/T 5X | 20.65 | 79.35 | 0 | 2.23 | 97.77 | 0 |

### 2.6 Non-Specific Binding Test by BVP ELISA

BVPs were incubated on ELISA plates by adding 1% BVP stock. After blocking, primary antibodies (i.e. test antibodies) was added and incubated, and anti-human-IgG-HRP was used for detection. BVP score was determined by normalizing absorbance by control wells with no test antibody. As shown in Table 8, the BVP score of V0013 and BMK1 were both acceptable, meaning low non-specific binding risk.

TABLE 8
BVP score of IL-15 heterodimer fusion protein

| IL-15 variants | BVP score |
|---|---|
| W327151-TXU1.P1-V0013.uIgG4V322 | 1.167 |
| W327151-BMK1 | 1.092 |

### 2.7 Ki-67 Proliferation Assays in Human/Cyno/Mouse T/NK Cells

Human PBMCs of healthy donor were purchased from OriBiotech and cultured in RPMI 1640 medium (Gibco, Cat.22400105) supplemented with 10% fetal bovine serum and 1% penicillin/streptomycin. Cynomolgus PBMCs were isolated from whole blood with Lymphoprep (STEMCELL, Cat. 07851) and cultured in RPMI 1640 medium supplemented with 10% fetal bovine serum and 1% penicillin/streptomycin. Mouse spleen cells were isolated from wild type C57BL/6 mice and cultured in RPMI 1640 medium (Gibco, Cat. A1049101) supplemented with 10% fetal bovine serum, 1% penicillin/streptomycin and 0.05 mM 2-mercaptoethanol.

Human PBMCs were treated with IL-15 variants at the indicated concentrations. 4 days after treatment, the PBMCs were stained with anti-CD3, anti-CD8 and anti-CD56 to gate for CD8+ T cells (CD3+/CD8+) and NK cells (CD3−/CD56+). Ki67 is a protein strictly associated with cell proliferation, and staining for intracellular Ki67 was performed using anti-Ki67 and Foxp3/Transcription Factor Staining Buffer Set. Lymphocytes were first gated on the basis of SSC and FSC. The lymphocytes were then gated based on CD3, CD8 and CD56 expression to identify CD8+T and NK cells. The percentage of Ki67 on CD8+T and NK cells was measured using FACS.

Cynomolgus PBMCs were treated with IL-15 variants at the indicated concentrations. 4 days after treatment, the PBMCs were stained with Abs against cyno CD3, CD8 and CD16 to gate for CD8+ T cells (CD3+/CD8+) and NK cells (CD3−/CD16+), and then stained for intracellular Ki67 using anti-Ki67 and Foxp3/Transcription Factor Staining Buffer Set. The percentage of Ki67 on CD8+T and NK cells was measured using FACS.

Mouse spleen cells were collected and treated with IL-15 variants at the indicated concentrations. 4 days aftertreatment, the cells were stained with Abs against mouse CD3, CD8 and CD335, to gate for CD8+ T cells (CD3+/CD8+) and NK cells (CD3−/CD335+), and then stained for intracellular Ki67 using anti-Ki67 and Foxp3/Transcription Factor Staining Buffer Set. The percentage of Ki67 on CD8+T and NK cells was measured using FACS.

To determine the effects of V0013 on CD8⁺ T and NK cells of human PBMCs, human PBMCs of healthy donor were cultured with V0013, V0016, V0017, V0018, W327151-BMK1, W327151-BMK2 and human IgG isotype control. The Ki67 level was used to measure proliferation of CD8⁺ T and NK cells.

The results demonstrated that W327151-BMK2 was most potent on human CD8⁺ T (FIG. 2A) and NK cell proliferation (FIG. 2B), while V0013 mildly promoted human CD8⁺ T and NK cell proliferation with a potency comparable to W327151-BMK1 (XENP24306) (Table 9). V0016, V0017 and V0018 induced over 1000-fold potency reduction compared to BMK2.

TABLE 9
Potency of IL-15 variants on human CD8+T and NK proliferation

| | Human CD8⁺ T | | Human NK | |
|---|---|---|---|---|
| | | Reduction | | Reduction |
| | EC₅₀ (nM) | (fold) | EC₅₀ (nM) | (fold) |
| W327151-TXU1.P1- | 18.75 | 334.8 | 8.91 | 742.5 |
| V0013.uIgG4V322 | | | | |
| W327151-TXU1.P1- | 88.13 | 1574 | 16.23 | 1353 |
| V0016.uIgG4 V322 | | | | |
| W327151-TXU1.P1- | >1000 | >17000 | 298.3 | 24858 |
| V0017.uIgG4 V322 | | | | |
| W327151-TXU1.P1- | >1000 | >17000 | 18.73 | 1561 |
| V0018.uIgG4 V322 | | | | |
| W327151-BMK1 | 93.28 | 1665.7 | 8.542 | 711.8 |
| W327151-BMK2 | 0.056 | 1.0 | 0.012 | 1.0 |

In order to explore the potency of IL-15 variants on cynomolgus CD8⁺ T and NK cells, cynomolgus PBMCs were cultured with W327151-TXU1.P1-V0013.uIgG4V322 and W327151-BMK2. As shown in FIG. 3, comparing with wt IL-15 (W327151-BMK2), W327151-TXU1.P1-V0013.uIgG4V322 mildly promoted proliferation of cynomolgus CD8⁺ T and CD16⁺ NK cells, with a substantially reduced potency of hundreds of folds, showing a similar pattern as on human T/NK cells.

Mouse spleen cells were also used to determine the effects of IL-15 variant W327151-TXU1.P1-V0013.uIgG4V322 on mouse CD8⁺ T and NK cells. After 4 days of incubation with V0013, W327151-BMK1, W327151-BMK2 and human IgG isotype control, percentage of Ki67⁺ of CD3⁺ CD8a⁺ or CD3-CD335⁺ cells were analyzed by flow cytometry to measure CD8⁺ T cell or NK cell proliferation, respectively.

As shown in FIG. 4, only W327151-BMK2 and V0013 could proliferate mouse CD8⁺ T (FIG. 4A) and NK cells (FIG. 4B), while BMK1 (XENP24306) was totally inactive. V0013 showed better potency in mouse NK than CD8+T. Furthermore, V0013 showed a similar degree of potency reduction in stimulating mouse T/NK proliferation as in human, suggesting the feasibility of using mouse tumor models to measure the in vivo efficacy of this IL-15 variants.

TABLE 10
The efficacy summary of IL-15 variants on human/cyno/mouse cell proliferation

| | Human | | Cynomolgus | | Mouse | |
|---|---|---|---|---|---|---|
| | CD8⁺ T cells | NK cells | CD8⁺ cells | CD16⁺ cells | CD8⁺ T cells | NK cells |
| | EC₅₀ (nM) | EC₅₀ (nM) | EC₅₀ (nM) | EC₅₀ (nM) | EC₅₀ (nM) | EC₅₀ (nM) |
| W327151-TXU1.P1- | 18.75 | 8.91 | 9.92 | 29.41 | / | 35.93 |
| V0013.uIgG4V322 | | | | | | |
| W327151-BMK1 | 93.28 | 8.542 | >100 | 14.75 | / | / |
| W327151-BMK2 | 0.056 | 0.012 | 0.58 | 0.07 | 3.97 | 0.49 |

### 2.8 Binding Assays in HH Cell Line

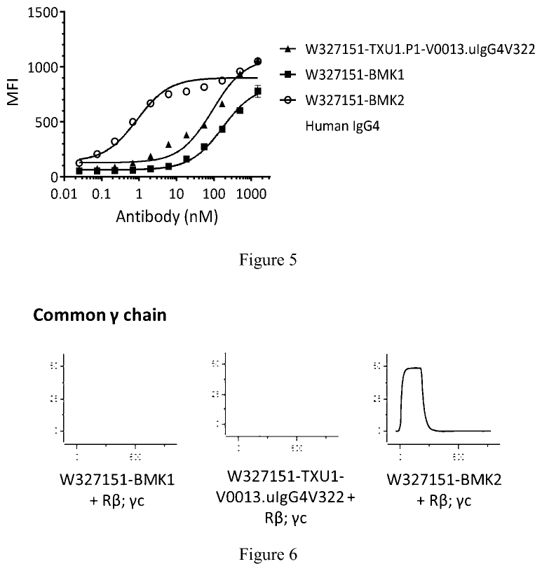

The relative binding of W327151-TXU1.P1-V0013.uIgG4V322 to the human lymphoma (HH) cell line was measured. As can be seen in FIG. 5, W327151-TXU1.P1-V0013.uIgG4V322 (EC50=91.89 nM), W327151-BMK1 (EC50=171.70 nM) and W327151-BMK2 (EC50=0.89 nM) bound to the HH cell line in a dose-dependent manner.

### 2.9 Receptor Binding Test by SPR

IL-15 variants binding affinity to its receptor were detected by SPR using Biacore 8K. Each variant was captured on anti-human Fc immobilized CM5 sensor chip (cytiva). W327151-hPro2.ECD.His at different concentrations were injected over the sensor chip at a flow rate of 30 uL/min for an association phase of 120 s, followed by 120 s dissociation. The chip was then regenerated by 10 mM Glycine-HCl pH 1.5 after each binding cycle.

As summarized in Table 11, V0013 retained receptor β binding affinity compared to BMK2 (wt IL-15), and showed no or weak binding to common γc. W327151-BMK1 showed affinity attenuation to receptor β and common γc. For common γc binding, V0013 was much weaker than BMK1 in FIG. 6. As a result, V0013 was a non-common γc binding IL-15.

TABLE 11
Affinity of IL-15 heterodimer fusion protein to IL-15Rβ and common γ_{c} by SPR

| | Receptor β | | | Common γ_{c} | | |
|---|---|---|---|---|---|---|
| IL-15 | kₐ (1/Ms) | k_{d} (1/s) | K_{D} (M) | ka (1/Ms) | kd (1/s) | K_{D} (M) |
| W327151-BMK2 | 7.76E+05 | 2.61E−03 | 3.36E−09 | 5.66E+03 | 4.24E−02 | 7.49E−06 |
| W327151-TXU1.P1- | 6.12E+05 | 4.83E−03 | 7.90E−09 | no or weak binding | | |
| V0013.uIgG4V322 | | | | | | |

| | | | |
|---|---|---|---|
| W327151-BMK1 | NA | 8.58E−06 | weak binding |

### 2.10 Non-Specific Binding Assay

The full panel non-specific binding assay was operated on a series of tumor or engineering cell lines, and detected by flow cytometry. There's no detectable binding of V0013 on cell lines (Table 12), meaning no non-specific binding risk.

TABLE 12
Binding summary in different cell lines

| | W327151-TXU1.P1- | |
|---|---|---|
| | V0013.uIgG4V322 | W327151-BMK1 |
| Cell | MFI | |
| Jurkat.2B8 | 20.1 | 24.7 |
| MDA-MB-453 | 23.3 | 23.4 |
| HT29 | 20.5 | 31.3 |
| KYSE-150 | 29.1 | 75.7 |
| HepG2 | 28.1 | 29.7 |
| SK-OV-3 | 40.6 | 181 |
| Calu-6 | 23.2 | 28.2 |
| BxPC-3 | 23 | 42.3 |
| DU145 | 54.1 | 19.6 |
| A375 | 15.9 | 14.1 |
| A204 | 28.5 | 75.5 |
| NCI-N87 | 23.8 | 41.6 |
| 8505C | 26.8 | 67.9 |
| H1 Hela | 19 | 22.2 |
| BT474 | 21.7 | 21.9 |
| Calu-3 | 20 | 38.8 |
| 293F | 19.2 | 28.5 |
| CHO-K1 | 22.7 | 44.8 |

### 2.10 Allogeneic Mixed Lymphocyte Reaction Assay

For human allogeneic mixed lymphocyte reaction, purified CD4⁺ T cells were co-cultured with allogeneic dendritic cells, and various concentrations of antibodies were added into the culture system. After incubation for 3 days, IFN-γ was detected by ELISA.

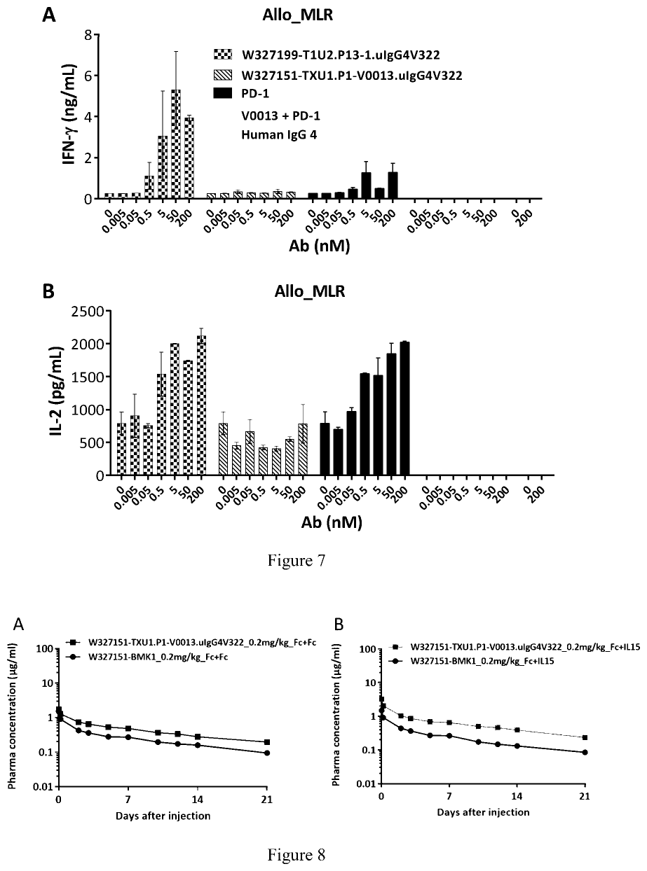

As shown in FIG. 7A, W327199-T1U2.P13-1.uIgG4V322 promoted the IFN-γ secretion in a dose-dependent manner, which was much more potent than that of the PD-1 antibody (W3056-AP17R1-2H2-Z1-R1-14A1-FC(IgG4.SP), US20200002420A1) or the combination treatment of W327151-TXU1.P1-V0013.uIgG4V322 and the PD-1 antibody. In IL-2 secretion assay, the potency of W327199-T1U2.P13-1.uIgG4V322 was comparable to the PD-1 antibody or combination group (FIG. 7B).

### Example 3

### In Vivo Characterization

### 3.1 Animals

All the procedures related to animal handling, care and the treatment in the study were performed according to the guidelines approved by the Institutional Animal Care and Use Committee (IACUC) of WuXi Biologics following the guidance of the Association for Assessment and Accreditation of Laboratory Animal Care (AAALAC).

### 3.2 Pharmacokinetics (PK)

The female C57BL/6 mice (8 to 9 week) from Charles River were randomly assigned to two dosing groups, and injected intravenously with IL-15 variants at dose of 0.2 mg/kg on day 0. Sample collection for immunology and pharmacokinetics analysis was also performed. The quarantine period was 21 days (prior to pre-dose period). The pre-dose period was 1 day prior to the initiation of dosing. Intravenous administration is the intended clinical route of administration. The systemic exposure to the test article was assessed in all mice. Blood samples were collected at pre-dose (Day 0) and 15 min, 4h, day2 (48h), day3 (72h), day5 (120h), day7 (168h), day10 (240h), day12 (288h), day14 (336h) and day21 (504h) after dosing. At each time point, approximately 0.03 mL blood was collected from the saphenous vein or other suitable site to harvest serum. Extreme care was taken during blood collection. Blood samples were collected into tubes without additive and placed on ice until they were processed. All samples were processed within 2 hours of collection.

### Measurement of Drug Serum Concentration by ELISA

Drug serum concentrations were measured by ELISA. Briefly, ELISA plate was coated with 1 μg/mL goat anti-human IgG Fc (Southern Biotech, 2049-01). After washing and blocking, serial diluted serum samples were added and then biotin labeled goat anti-human IgG Fc (Southern Biotech, SB-2049-08) or anti-human IL-15 (R&D, BAM247) was used as detection antibody. HRP-Streptavidin (Thermo-21127) and TMB substrate (Life Technologies, 002023) were used for color development. The absorbance of the wells was measured at (450-540) nm with a multiwall plate reader (SpectraMax® M5e). Generate standard curve according to the standard samples, and analyze serum samples with SoftMax.

### Immunogenicity (ADA)

Mouse serum was harvested at day5, day7, day14, day21 after dosing. Anti-drug antibody (ADA) production was measured by ELISA. ELISA plate was coated with 1 μg/mL W327151-TXU1.P1-V0013.uIgG4v322 & W327151-BMK1. After washing and blocking, serial diluted serum samples were added and then goat anti-mouse IgG Fc fragment cross-adsorbed HRP (BETHYL, A90-231P) was used as detection antibody. Then TMB substrate (Life Technologies, 002023) were used for color development. The absorbance was measured as above. Serum of pre-dosing was used as baseline.

### Data Analyses

The data was evaluated for treatment-related effects by making comparisons of individual value, and between intervals where appropriate. The significance of any findings was determined based on prior experience of the scientists involved. The serum concentration was subjected to a non-compartmental pharmacokinetic analysis by using the Phoenix WinNonlin software (version 8.1, Pharsight, Mountain View, CA). The linear/log trapezoidal rule was applied in obtaining the PK parameters, data was expressed with Mean±SD.

As shown in FIG. 8 and Table 13, potency reduction in V0013 and BMK1 (XENP24306) obviously led to extended serum half-life compared to wild-type IL-15 (t_{1/2} ˜0.5 day, US2018118805A1). V0013 and BMK1 showed similar C₀ and t_{1/2}. Additionally, V0013 showed less clearance and better drug exposure (AUC) than BMK1, without causing toxicity and nearly no ADA production (Table 14). Prolonged PK profile will likely contribute to extended drug exposure and improved pharmacodynamics.

As shown in FIGS. 8A and B, plasma drug concentration detected by different detection methods, either Fc-Fc or IL-15-Fc, was consistent, suggesting good in vivo stability of the IL-15 variant tested.

TABLE 13
PK parameters determined by non-compartmental analysis (WinNonlin)

| | PK method | | | |
|---|---|---|---|---|
| | Fc-Fc | | Fc-IL15 | |
| IL-15 variants | BMK1 | V0013 | BMK1 | V0013 |
| t_{1/2} (h) | 229 | 253 | 248 | 227 |
| Cₘₐₓ (μg/mL) | 1.55 | 1.76 | 1.53 | 3.39 |
| AUC₀₋ₜ (hm * μg/mL) | 125 | 219 | 118 | 304 |
| Cl_obs (mL/day/kg) | 30.8 | 16.5 | 32.2 | 12.6 |

TABLE 14
ADA production determined by ELISA

| | Group | | | | | | | |
|---|---|---|---|---|---|---|---|---|
| | W327151-TXU1.P1- | | | | W327151-BMK1, | | | |
| | V0013.uIgG4v1, 0.02 mg/kg | | | | 0.02 mg/kg | | | |
| Animal ID | 126# | 144# | 121# | 136# | 133# | 132# | 119# | 143# |
| Day 5 | − | − | − | − | − | − | − | − |
| Day 7 | − | − | − | − | − | − | − | − |
| Day 14 | + | − | − | − | − | − | − | − |
| Day 21 | − | + | − | − | − | − | − | − |

ADA Criteria: OD ratio (S/N) <2, −; 2~5, +; 5~10, ++.

### 3.3 Mouse Maximum Tolerance Dose (MTD)

The female C57BL/6 mice (8 to 9 week) from Charles River were randomly assigned to different groups, and injected intravenously with IL-15 variants at different doses every 3 days (Q3D). Mice were monitored after dosing, and body weight was collected at every 3 days.

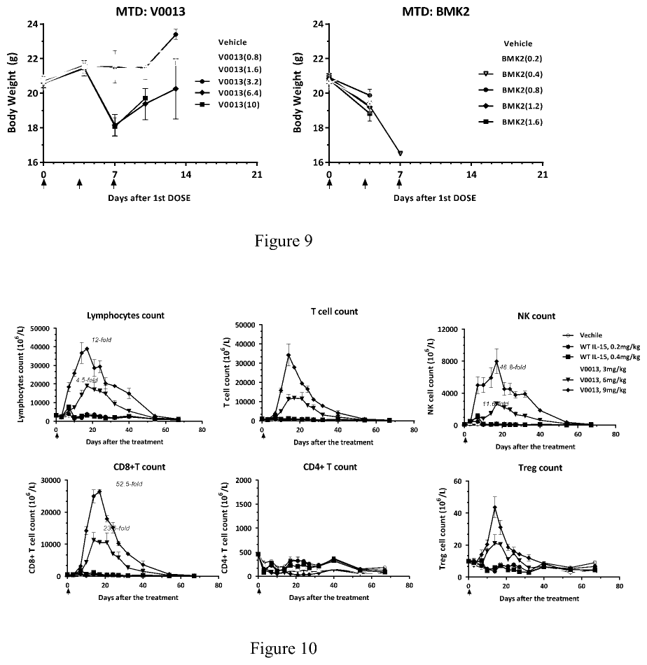

As shown in FIG. 9, mice treated with 0.8-3.2 mg/kg V0013 were well tolerated, as no body weight loss. Dosing of 6.4 and 10 mg/kg V0013 (Q3D) induced separate mouse died, and left mice endured with over 10% body weight loss at day 7. WT IL-15 (BMK2) was more toxic compared to V0013, only mice in 0.2 mg/kg treatment group (Q2D) were survival. As a result, V0013 achieved 16-fold MTD (3.2 mg/kg, Q3D) improvement than WT IL-15 (0.2 mg/kg, Q3D).

### 3.4 Pharmacodynamics (PD)

Pharmacodynamic of IL-15 variant was determined on female C57BL/6 mice, due to mouse-cross reactivity of V0013. Mice were treated with V0013 or WT IL-15 in different doses, and the blood was collected for analysis at different time points post dosing. Peripheral lymphocytes were determined by flow cytometry. The pre-dose period was 1 day prior to the initiation.

In FIG. 10, V0013 expanded peripheral lymphocytes, especially CD8+T and NK cells. Here, NK cells were more sensitive to V0013 with a long-acting response (from day 3 to day 57) than WT IL-15 (day 7-10). The maximum expansion of CD8+T and NK cell induced by V0013 was at day 17, suggesting the extended PD effect compared to WT IL-15 (day 7). Furthermore, the proliferation of CD8+T and NK cells were both dose response. Besides, Treg was slightly expanded by V0013 in high dose, while CD4+ T cells were unresponsive.

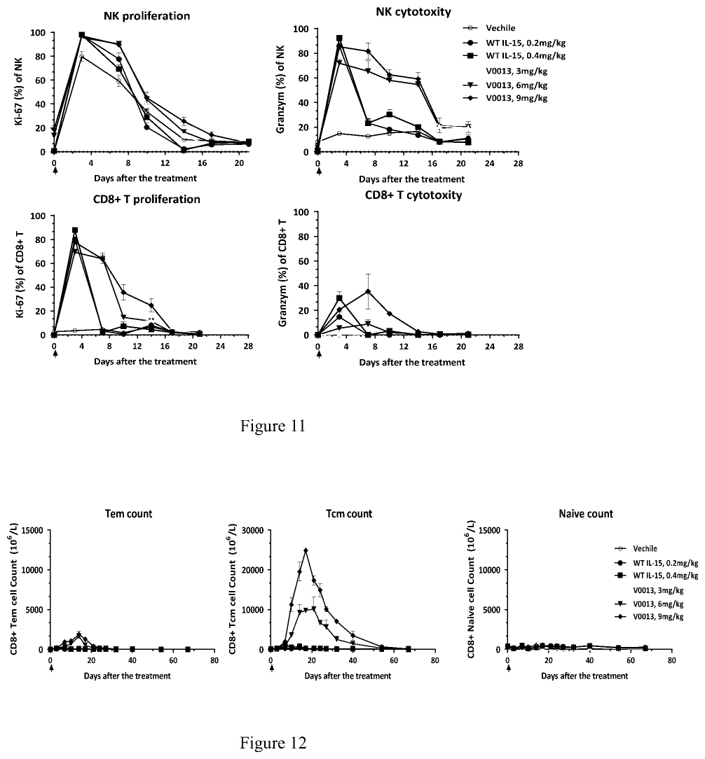

For detail activation of NK and CD8+ T cell, V0013 or WT IL-15 induced more longer proliferation (Ki-67) period of NK than CD8+T (FIG. 11). The cytotoxicity of NK, determined by Granzyme B expression, was stronger and more lasting than CD8+T. As a conclusion, V0013 and WT IL-15 induced long-acting PD in NK than CD8+T.

Meanwhile, CD44 and CD62L were used to distinguish memory and naïve subtypes of CD8+ T cell. As collected in FIG. 12, V0013 mainly induced center memory CD8+T (Tcm) proliferation, and slightly expended effector memory CD8+T (Tem), not naïve CD8+T.

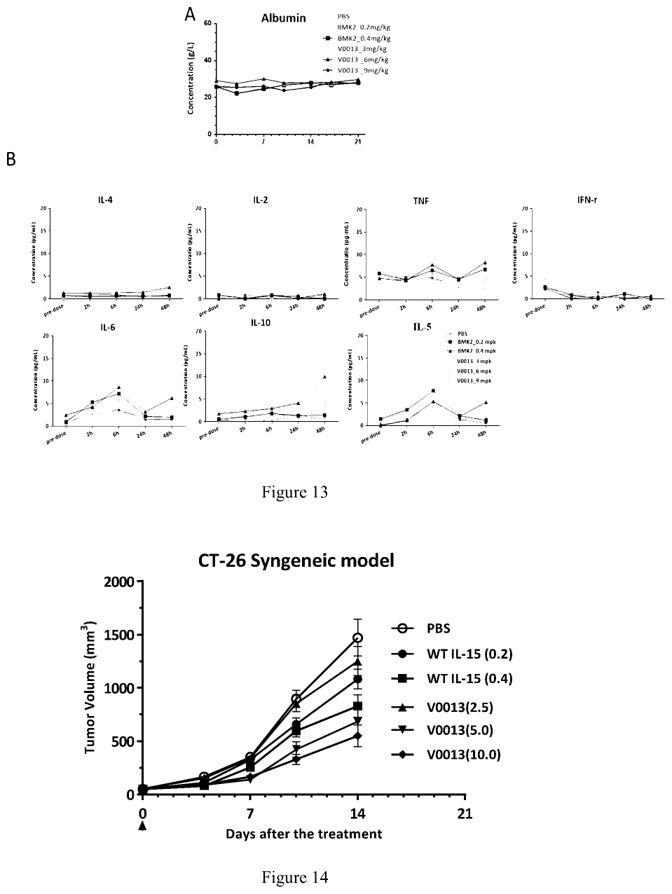

During PD period, there were no peripheral albumin loss, or strong cytokine release (FIG. 13).

As a result, V0013, a weak IL-15 with limited adverse events, induced better CD8+T and NK cell activation and proliferation, and induced memory subtypes than WT IL-15. The extended and enhanced PD could dominate to better anti-tumor efficiency.

### 3.4 Anti-Tumor Efficiency

CT26 syngeneic, Daudi CDX and A375 humanized models were used for efficacy study. The CT26 cells were implanted (s.c.) into the right flanks of Balb/c mice. The Daudi cells implanted (s.c.) into the right flanks of SCID mice. The A375 cells implanted (s.c.) into the right flanks of NPG mice, followed by human PBMC transplanting through i.p. When tumors reach corresponding volume, tumor bearing mice were randomly divided into different groups, dosing with IL-15 variant. Tumor sizes were measured in two dimensions using a caliper, and the volume was expressed in mm³. Results were represented by mean and the standard error (Mean±SEM). Statistical analysis was operated by two-way ANOVA, P<0.05 was considered to be statistically significant.

In CT26 syngeneic model (FIG. 14), treated with V0013 at 2.5/5/10 mg/kg showed significant anti-tumor effect comparing to the treatment of PBS, the TGI was 15.6%, 55.6% and 64.9% specially. WT IL-15 showed similar dose-response. Compared with WT IL-15, V0013 showed better anti-tumor effect in MTD dose. The efficiency of V0013 was dominated by CD8+T and NK cells.

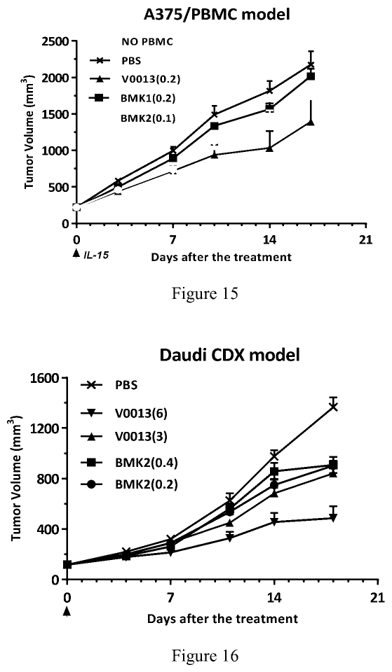

A375 humanized model were used to determine V0013 effect on T cells, where human T cells were reconstructed. As shown in FIG. 15, V0013 significantly inhibited tumor growth as a single agent, which was better than BMK2 and BMK1. Human T cell activation and proliferation by V0013 dominate to better anti-tumor effect.

The Daudi CDX model was used to determine NK efficiency. In FIG. 16, BMK2 showed limited anti-tumor effect, even in high dose (0.4 mg/kg). In comparation, V0013 showed significant anti-tumor effect and dose response. That means, V0013 induced mouse NK cell expansion and activation dominate to anti-tumor effect.

As a result, V0013 showed predominant effect than WT IL-15, no matter by CD8+T or NK cells. Pro-longed PK, enhanced PD of V0013 induced better efficacy.

Those skilled in the art will further appreciate that the present invention may be embodied in other specific forms without departing from the spirit or central attributes thereof. In that the foregoing description of the present disclosure provides only exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

1-44. (canceled)

45. A polypeptide complex comprising an Interleukin-15 (IL-15) variant domain, an IL-15Rα domain, a first dimerization domain and a second dimerization domain, wherein:
the IL-15 variant domain comprises an IL-15 variant peptide of the amino acid sequence of any of SEQ ID Nos: 51-69; and
the first dimerization domain and the second dimerization domain associates together to form a dimer.

46. The polypeptide complex of claim 45, wherein the IL-15Rα domain comprises or consists of a wild-type IL-15Rα protein, an IL-15Rα variant or any fragment thereof that retains IL-15 binding activity, such as a fragment as set forth in SEQ ID No: 2.

47. The polypeptide complex of claim 46, further comprising one or more antigen-binding moieties, optionally in VHH, Fab, scFv, VH or VL format.

48. The polypeptide complex of claim 47, wherein the antigen-binding moiety specifically binds to a target antigen selected from PD-1, PD-L1, PD-L2, CTLA-4, LAG3, TIM-3, TIM4, 4-1BB, OX-40, OX-40L, GITR, A2aR, TIGIT, CD96, PVRIG, CD226, 5T4, VISTA, VSIG3, VSIG4, ICOS, CD28, CD3, CD4, CD8, CD45, CD44v6, CD27, CD47, SIRPAα, SLAMF7, CD24, Siglec10, Siglec15, Siglec8, VSIR, VSIG4, PSGL-1, C5AR1, BTN1A1, BTN3A1, CD70, RANKL, CSF1R, CSF2RB, TNFRSF1/1a/1b, BDCA2, BTLA, C5aR, NKG2A, NKG2D, NKp30, NKp46, CD16a, CD56, CD166, FCGR3, CD2, Neurophilin-1, CCR8, CCR2, CCR4, CCR5, CCR6, CCR7, CCR8, GCGR, CXCR2, CXCR4, CXCR5, CALCRL, ETAR, GLP1R, CX3CR1, GPR1, GPR17, GPR20, GPR30, GPR34, GPR-65, GPCR78, GPRC5D, GPR84, LGR4, LGR5, VEGF, VEGFR, HER2, HER3, Trop2, pCAD, ERα, EGFR, de2-7 EGFR, EGFRvIII, PSMA, PSCA, PSA, TAG-72, SEZ6, SEZ6L, SEZ6L2, SEMA4D, DLL3, GD2, GPC3, KLB, KLRB1, KLRG1, GPC1, PCSK9, EpCAM, p-Cadherin, Caludin 6, Caludin 18.2, FGFR2b, FGFR3, FGFR4, MUC1, MUC13, MUC16, MUC17, MUCL3, FolRa, TfR, TF, TFR, TFPI, c-Met, NY-ESO-1, GUCY2C, LIV-1, Integrin αvβ6, Integrin α10β1, Integrin α3, Integrin α5β4, Integrin αvβ, Integrin αvβ8, ROR1, ROR2, PRLR, PTK7, B7-H3, Nectin-4, NetG1, Ax1, CD147, LRRC15, Napi2b, STEAP1, LY6G6D, LYPD1, MACRO, MerTK, MICA, MICB, MSLN, Mkars, G12D, CDH3, CDH6, CDH17, APLA2, CAIX, CD46, CD47, CLDN6, EphA3, Fucosyl-GM1, ITGA3, Kallikrein, MISRII, Podocalyxin, RON, ROBO1, PAUF, PLA2, Podocalyxin, PRLR, PTK7, TM4SF1, TMEFF2, TREAKR, TREM-1, TREM-2, uPARAP, TYRP1, KAAG1, RU2AS, CD146, CD63, Endoglin, Globo H, IGF-1R, TEM1, TEM8, TAX1BP3, ADAM-9, ENPP3, EphA2, EphA3, FcRH5, NaPi3b, TWEAK, DLK1, SORT1, SSTR2, STEAP1, CD25, CD39, GARP, LRRC33, LAIR1, LAMP3, LAP, LEPR, LILRB1, LILRB2, LILRB4, RAGE, FGL1, TPBG, PDGFRB, TGFBR2, CEACAM1, CEACAM5, CEACAM6, Carcinoembryonic antigen (CEA), ICAM1, A33, CAMPATH-1 (CDw52), Carboanhydrase IX (MN/CA IX), CD248, PDPN, ITGB1, ITGAV, CD20, CD19, CD21, CD22, CLL, BCMA, DCLK1, DDR1, DLK1, DPEP3, DKK1, CD5, CD13, CD30, CD33, CD34, CD36, CD37, CD38, CD43, CD52, CD55, CD94, CD99, CD7, CD71, CD73, CD74, CD79a, CD79b, CD229, CD132, CD133, G250, CSF1R (CD115), HLA-DR, HLA-G, HTRA1, TRA-1-60, IGFR, IL-2 receptor, MCSP (Melanoma-associated cell surface chondroitin sulphate proteoglycan), ART1, ASGR1, B7H3, B7-H4, B7H6, CD124, c-Kit (CD117), CD7, Clex12A, Clever-1, IL-13RA2, IL-11RA, IL-31RA, IL-4RA, IFNAR, ActRIIb, IL-7R, SLAMF7, Fms-like tyrosine kinase 3 (FLT-3, CD135), GFRA1, BTLA, GloboH, CSF2RB, chondroitin sulfate proteoglycan 4 (CSPG4), ITGA4, Clec5a, Clec7a, Clec9a, Clec12a, CLEC14, CD205, CD206, CD200R1, CD228, CD229, CD40, CD40L, FcRn, TLR8, TLR9, TNFR2, LTBR, CD44, CD93, PDGF, PDGFR-alpha (CD140a), PDGFR-beta (CD140b), CD146, CD147, CRTH2, TNF-α, TGF-β, IL1RAcP, TSLP, DR5, ST2, fibroblast activating protein (FAP), CDCP1, Derlin1, Tenascin, frizzled 1-10, the vascular antigens VEGFR2 (KDR/FLK1), VEGFR3 (FLT4, CD309), Endoglin, Tie2 and other TAAs, I/O checkpoints, tumor microenvironment targets, or autoimmune and inflammatory diseases associated targets.

49. The polypeptide complex of claim 47, wherein the first and second antigen-binding moieties are in VHH format and specifically bind to PD-1, optionally the first and second antigen-binding moieties have same amino acid sequences as set forth in SEQ ID No: 80.

50. The polypeptide complex of claim 45, wherein the first dimerization domain is one chain of an immunoglobulin Fc region, and the second dimerization domain is the other chain of the immunoglobulin Fc region, wherein the Fc region comprises a human IgG Fc such as a human IgG1 Fc, IgG2 Fc, IgG3 Fc, IgG4 Fc, or a variant thereof.

51. The polypeptide complex of claim 50, wherein the Fc variant comprises:
(a) a human IgG4 Fc engineered to comprise one or more of the following: S228P mutation, F234A/L235A mutations, M252Y/S254T/T256E mutations and “knob into hole” structure; or
(b) a human IgG1 Fc engineered to comprise one or more of the following: M252Y/S254T/T256E mutations, G236R/L328R mutations and “knob into hole” structure.

5. The polypeptide complex of claim 51, comprising a first chain and a second chain, wherein:
(a) the first chain comprises an amino acid sequence as set forth in SEQ ID No: 19, and the second chain comprises an amino acid sequence as set forth in SEQ ID No: 20;
(b) the first chain comprises an amino acid sequence as set forth in SEQ ID No: 70, and the second chain comprises an amino acid sequence as set forth in SEQ ID No: 71;
(c) the first chain comprises an amino acid sequence as set forth in SEQ ID No: 72, and the second chain comprises an amino acid sequence as set forth in SEQ ID No: 73;
(d) the first chain comprises an amino acid sequence as set forth in SEQ ID No: 74, and the second chain comprises an amino acid sequence as set forth in SEQ ID No: 75;
(e) the first chain comprises an amino acid sequence as set forth in SEQ ID No: 21, and the second chain comprises an amino acid sequence as set forth in SEQ ID No: 22;
(f) the first chain comprises an amino acid sequence as set forth in SEQ ID No: 23, and the second chain comprises an amino acid sequence as set forth in SEQ ID No: 24;
(g) the first chain comprises an amino acid sequence as set forth in SEQ ID No: 25, and the second chain comprises an amino acid sequence as set forth in SEQ ID No: 26;
(h) the first chain comprises an amino acid sequence as set forth in SEQ ID No: 76, and the second chain comprises an amino acid sequence as set forth in SEQ ID No: 77;
(i) the first chain comprises an amino acid sequence as set forth in SEQ ID No: 13, and the second chain comprises an amino acid sequence as set forth in SEQ ID No: 14;
(j) the first chain comprises an amino acid sequence as set forth in SEQ ID No: 15, and the second chain comprises an amino acid sequence as set forth in SEQ ID No: 16;
(k) the first chain comprises an amino acid sequence as set forth in SEQ ID No: 17, and the second chain comprises an amino acid sequence as set forth in SEQ ID No: 18;
(l) the first chain comprises an amino acid sequence as set forth in SEQ ID No: 39, and the second chain comprises an amino acid sequence as set forth in SEQ ID No: 40;
(m) the first chain comprises an amino acid sequence as set forth in SEQ ID No: 41, and the second chain comprises an amino acid sequence as set forth in SEQ ID No: 42;
(n) the first chain comprises an amino acid sequence as set forth in SEQ ID No: 43, and the second chain comprises an amino acid sequence as set forth in SEQ ID No: 44;
(o) the first chain comprises an amino acid sequence as set forth in SEQ ID No: 45, and the second chain comprises an amino acid sequence as set forth in SEQ ID No: 46;
(p) the first chain comprises an amino acid sequence as set forth in SEQ ID No: 47, and the second chain comprises an amino acid sequence as set forth in SEQ ID No: 48; or
(q) the first chain comprises an amino acid sequence as set forth in SEQ ID No: 49, and the second chain comprises an amino acid sequence as set forth in SEQ ID No: 50.

53. A fusion protein, comprising the IL-15 variant peptide as defined in claim 45 operably linked to a non-IL-15 moiety, wherein the non-IL-15 moiety is a moiety that can enhance the half-life of the fusion protein in vivo or an IL-15Rα domain, optionally the IL-15Rα domain comprises or consists of a wild-type IL-15Rα protein, an IL-15Rα variant or any fragment thereof that retains IL-15 binding activity.

54. An IL-15 variant peptide consisting of the amino acid sequence of any of SEQ ID Nos: 51-69.

55. A nucleic acid molecule, comprising a nucleic acid sequence(s) encoding the first and second chain of the polypeptide complex of claim 45.

56. A host cell or a vector comprising the nucleic acid molecule of claim 55.

57. A pharmaceutical composition comprising the polypeptide complex of claim 45, as well as a pharmaceutically acceptable carrier.

58. An immunoconjugate comprising the polypeptide complex of claim 45 conjugated to a moiety selected from a chemical compound (including a cytotoxic agent, a cytostatic agent), polypeptide, carbohydrate, lipid, nucleic acid or a combination thereof.

59. A method for producing the polypeptide complex of claim 45, comprising the steps of:
culturing a host cell comprising a vector(s) encoding one or both chains of the polypeptide complex; and
isolating the polypeptide complex from the host cell culture.

60. A method for treating or preventing cancer, an infectious disease, an immunodeficiency or lymphopenia in a subject, comprising administering an effective amount of the polypeptide complex of claim 45 to the subject.

61. The method of claim 60, which further comprises administering an additional anti-tumor therapy, such as cell therapy, targeted therapy, chemotherapy and gene therapy.

62. The method of claim 60, wherein the cancer is selected from breast cancer, lung cancer, colon cancer, ovarian cancer, melanoma, bladder cancer, renal cell carcinoma, liver cancer, prostate cancer, stomach cancer, pancreatic cancer, lymphoma, leukemia, and multiple myeloma.

63. A method for treating or preventing cancer, an infectious disease, an immunodeficiency or lymphopenia in a subject, comprising administering a cell therapy (including tumor-infiltrating lymphocyte (TIL) therapy, T cell receptor (TCR) therapy, chimeric antigen receptor (CAR) T cell therapy, and NK cell therapy) or a gene therapy (including using lentivirus, AAV, poxvirus, herpes zoster virus, oncolytic virus, or other RNA/DNA vectors for gene delivery), wherein the cell therapy and the gene therapy comprises delivering the nucleic acid molecule of claim 55 to the subject.

64. The method of claim 63, wherein the cancer is selected from breast cancer, lung cancer, colon cancer, ovarian cancer, melanoma, bladder cancer, renal cell carcinoma, liver cancer, prostate cancer, stomach cancer, pancreatic cancer, lymphoma, leukemia, and multiple myeloma.


SEQUENCE LISTING (80 sequences)

SEQ ID NO: 1 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 65 aa, synthetic construct
  REGION 1..65: note = Human wild-type IL-15R protein fragment
ITCPPPMSVEHADIWVKSYSLYSRERYICNSGFKRKAGTSSLTECVLNKATNVAHWTTPSLKCIR

SEQ ID NO: 3 - protein, 231 aa, synthetic construct
  REGION 1..231: note = 1st chain of the IgG1 Fc variant (V805)
EPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 231 aa, synthetic construct
  REGION 1..231: note = 2nd chain of the IgG1 Fc variant (V805)
EPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 5 - protein, 228 aa, synthetic construct
  REGION 1..228: note = 1st chain of the IgG4 Fc variant (V1)
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 6 - protein, 229 aa, synthetic construct
  REGION 1..229: note = 2nd chain of the IgG4 Fc variant (V1)
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 7 - protein, 229 aa, synthetic construct
  REGION 1..229: note = 1st chain of the IgG4 Fc variant (V322)
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 229 aa, synthetic construct
  REGION 1..229: note = 2nd chain of the IgG4 Fc variant (V322)
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 9 - protein, 345 aa, synthetic construct
  REGION 1..345: note = 1st chain of V0007.uIgG1V805
NWVNVISELKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSEPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 296 aa, synthetic construct
  REGION 1..296: note = 2nd chain of V0007.uIgG1V805
ITCPPPMSVEHADIWVKSYSLYSRERYICNSGFKRKAGTSSLTECVLNKATNVAHWTTPSLKCIREPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 11 - protein, 342 aa, synthetic construct
  REGION 1..342: note = 1st Chain of V0008.uIgG4V1
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - protein, 293 aa, synthetic construct
  REGION 1..293: note = 2nd chain of V0008.uIgG1V1
ITCPPPMSVEHADIWVKSYSLYSRERYICNSGFKRKAGTSSLTECVLNKATNVAHWTTPSLKCIRESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 13 - protein, 341 aa, synthetic construct
  REGION 1..341: note = 1st chain of V0010.uIgG1V1
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 14 - protein, 293 aa, synthetic construct; identical to SEQ ID NO: 12
  REGION 1..293: note = 2nd chain of V0010.uIgG4V1

SEQ ID NO: 15 - protein, 340 aa, synthetic construct
  REGION 1..340: note = 1st chain of V0011.uIgG1V1
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 16 - protein, 293 aa, synthetic construct; identical to SEQ ID NO: 12
  REGION 1..293: note = 2nd chain of V0011.uIgG4V1

SEQ ID NO: 17 - protein, 339 aa, synthetic construct
  REGION 1..339: note = 1st chain of V0012.uIgG1V1
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 18 - protein, 293 aa, synthetic construct; identical to SEQ ID NO: 12
  REGION 1..293: note = 2nd chain of V0012.uIgG4V1

SEQ ID NO: 19 - protein, 338 aa, synthetic construct
  REGION 1..338: note = synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 20 - protein, 293 aa, synthetic construct; identical to SEQ ID NO: 12
  REGION 1..293: note = synthetic construct

SEQ ID NO: 21 - protein, 337 aa, synthetic construct
  REGION 1..337: note = synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 22 - protein, 293 aa, synthetic construct; identical to SEQ ID NO: 12
  REGION 1..293: note = synthetic construct

SEQ ID NO: 23 - protein, 336 aa, synthetic construct
  REGION 1..336: note = synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 24 - protein, 293 aa, synthetic construct; identical to SEQ ID NO: 12
  REGION 1..293: note = synthetic construct

SEQ ID NO: 25 - protein, 335 aa, synthetic construct
  REGION 1..335: note = synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 26 - protein, 293 aa, synthetic construct; identical to SEQ ID NO: 12
  REGION 1..293: note = synthetic construct

SEQ ID NO: 27 - protein, 341 aa, synthetic construct
  REGION 1..341: note = synthetic construct
WVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 28 - protein, 293 aa, synthetic construct; identical to SEQ ID NO: 12
  REGION 1..293: note = synthetic construct

SEQ ID NO: 29 - protein, 340 aa, synthetic construct
  REGION 1..340: note = synthetic construct
VNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 30 - protein, 293 aa, synthetic construct; identical to SEQ ID NO: 12
  REGION 1..293: note = synthetic construct

SEQ ID NO: 31 - protein, 339 aa, synthetic construct
  REGION 1..339: note = synthetic construct
NVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 32 - protein, 293 aa, synthetic construct; identical to SEQ ID NO: 12
  REGION 1..293: note = synthetic construct

SEQ ID NO: 33 - protein, 338 aa, synthetic construct
  REGION 1..338: note = synthetic construct
VISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 34 - protein, 293 aa, synthetic construct; identical to SEQ ID NO: 12
  REGION 1..293: note = synthetic construct

SEQ ID NO: 35 - protein, 337 aa, synthetic construct
  REGION 1..337: note = synthetic construct
ISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 36 - protein, 293 aa, synthetic construct; identical to SEQ ID NO: 12
  REGION 1..293: note = synthetic construct

SEQ ID NO: 37 - protein, 336 aa, synthetic construct
  REGION 1..336: note = synthetic construct
SDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 38 - protein, 293 aa, synthetic construct; identical to SEQ ID NO: 12
  REGION 1..293: note = synthetic construct

SEQ ID NO: 39 - protein, 335 aa, synthetic construct
  REGION 1..335: note = synthetic construct
DLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 40 - protein, 293 aa, synthetic construct; identical to SEQ ID NO: 12
  REGION 1..293: note = synthetic construct

SEQ ID NO: 41 - protein, 334 aa, synthetic construct
  REGION 1..334: note = synthetic construct
LKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 42 - protein, 293 aa, synthetic construct; identical to SEQ ID NO: 12
  REGION 1..293: note = synthetic construct

SEQ ID NO: 43 - protein, 333 aa, synthetic construct
  REGION 1..333: note = synthetic construct
KKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 44 - protein, 293 aa, synthetic construct; identical to SEQ ID NO: 12
  REGION 1..293: note = synthetic construct

SEQ ID NO: 45 - protein, 332 aa, synthetic construct
  REGION 1..332: note = synthetic construct
KIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 46 - protein, 293 aa, synthetic construct; identical to SEQ ID NO: 12
  REGION 1..293: note = synthetic construct

SEQ ID NO: 47 - protein, 331 aa, synthetic construct
  REGION 1..331: note = synthetic construct
IEDLIQSMHIDATLYTESDVHPSCKVTAMKCFLLELQVISLESGDASIHDTVENLIILANNSLSSNGNVTESGCKECEELEEKNIKEFLQSFVHIVQMFINTSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 48 - protein, 293 aa, synthetic construct; identical to SEQ ID NO: 12
  REGION 1..293: note = synthetic construct

SEQ ID NO: 49 - protein, 330 aa, synthetic construct
  REGION 1..330: note = synthetic construct
EDLIQSMHIDATLYTESDVHPSCKVTAMKCFLLELQVISLESGDASIHDTVENLIILANNSLSSNGNVTESGCKECEELEEKNIKEFLQSFVHIVQMFINTSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 50 - protein, 293 aa, synthetic construct; identical to SEQ ID NO: 12
  REGION 1..293: note = synthetic construct

SEQ ID NO: 51 - protein, 110 aa, synthetic construct
  REGION 1..110: note = synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMF

SEQ ID NO: 52 - protein, 109 aa, synthetic construct
  REGION 1..109: note = synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQM

SEQ ID NO: 53 - protein, 108 aa, synthetic construct
  REGION 1..108: note = synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQ

SEQ ID NO: 54 - protein, 107 aa, synthetic construct
  REGION 1..107: note = synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIV

SEQ ID NO: 55 - protein, 113 aa, synthetic construct
  REGION 1..113: note = synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINT

SEQ ID NO: 56 - protein, 112 aa, synthetic construct
  REGION 1..112: note = synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIN

SEQ ID NO: 57 - protein, 111 aa, synthetic construct
  REGION 1..111: note = synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFI

SEQ ID NO: 58 - protein, 113 aa, synthetic construct
  REGION 1..113: note = synthetic construct
WVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 59 - protein, 112 aa, synthetic construct
  REGION 1..112: note = synthetic construct
VNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 60 - protein, 111 aa, synthetic construct
  REGION 1..111: note = synthetic construct
NVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 61 - protein, 110 aa, synthetic construct
  REGION 1..110: note = synthetic construct
VISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 62 - protein, 109 aa, synthetic construct
  REGION 1..109: note = synthetic construct
ISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 63 - protein, 108 aa, synthetic construct
  REGION 1..108: note = synthetic construct
SDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 64 - protein, 107 aa, synthetic construct
  REGION 1..107: note = synthetic construct
DLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 65 - protein, 106 aa, synthetic construct
  REGION 1..106: note = synthetic construct
LKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 66 - protein, 105 aa, synthetic construct
  REGION 1..105: note = synthetic construct
KKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 67 - protein, 104 aa, synthetic construct
  REGION 1..104: note = synthetic construct
KIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 68 - protein, 103 aa, synthetic construct
  REGION 1..103: note = synthetic construct
IEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 69 - protein, 102 aa, synthetic construct
  REGION 1..102: note = synthetic construct
EDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 70 - protein, 339 aa, synthetic construct
  REGION 1..339: note = synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 71 - protein, 294 aa, synthetic construct
  REGION 1..294: note = synthetic construct
ITCPPPMSVEHADIWVKSYSLYSRERYICNSGFKRKAGTSSLTECVLNKATNVAHWTTPSLKCIRESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 72 - protein, 339 aa, synthetic construct
  REGION 1..339: note = synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 73 - protein, 294 aa, synthetic construct
  REGION 1..294: note = synthetic construct
ITCPPPMSVEHADIWVKSYSLYSRERYICNSGFKRKAGTSSLTECVLNKATNVAHWTTPSLKCIRESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 74 - protein, 341 aa, synthetic construct
  REGION 1..341: note = synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFEPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 75 - protein, 296 aa, synthetic construct; identical to SEQ ID NO: 10
  REGION 1..296: note = synthetic construct

SEQ ID NO: 76 - protein, 486 aa, synthetic construct
  REGION 1..486: note = synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSGGGGSGGGGSGGGGSQVQLVESGGGVVQPGGSLRLSCAASDSIDSLVNMGWYRQAPGKQRELVALIATYITHYADFVKGRFTISRDNSKNTLYLQMNSLRAEDTAVYYCYARNIIVDYWGQGTLVTVS

SEQ ID NO: 77 - protein, 441 aa, synthetic construct
  REGION 1..441: note = synthetic construct
ITCPPPMSVEHADIWVKSYSLYSRERYICNSGFKRKAGTSSLTECVLNKATNVAHWTTPSLKCIRESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGGGGGSGGGGSGGGGSGGGGSGGGGSGGGGSGGGGSQVQLVESGGGVVQPGGSLRLSCAASDSIDSLVNMGWYRQAPGKQRELVALIATYITHYADFVKGRFTISRDNSKNTLYLQMNSLRAEDTAVYYCYARNIIVDYWGQGTLVTVS

SEQ ID NO: 78 - protein, 230 aa, synthetic construct
  REGION 1..230: note = synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDLEGGGGSGGGGHHHHHH

SEQ ID NO: 79 - protein, 249 aa, synthetic construct
  REGION 1..249: note = synthetic construct
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENLEGGGGSGGGGHHHHHH

SEQ ID NO: 80 - protein, 113 aa, synthetic construct
  REGION 1..113: note = synthetic construct
QVQLVESGGGVVQPGGSLRLSCAASDSIDSLVNMGWYRQAPGKQRELVALIATYITHYADFVKGRFTISRDNSKNTLYLQMNSLRAEDTAVYYCYARNIIVDYWGQGTLVTVS